SEQUENCE LISTING (3 sequences)

SEQ ID NO: 1 - DNA, 21 bp, Artificial Sequence
  random synthetic sequence
gggcgcgggatagggcgcggg

SEQ ID NO: 2 - DNA, 30 bp, Artificial Sequence
  random synthetic sequence
gggcgcgggaaataaaataaagggcgcggg

SEQ ID NO: 3 - DNA, 9 bp, artificial sequence
  random sequence
cccgcgccc

(19) United States
(12) Patent Application Publication
(10) Pub. No.: US 2004/0038264 A1
(43) Pub. Date: Feb. 26, 2004

(54) Fractal dimension analysis of nanoparticle aggregates using angle dependent light scattering for the detection and characterization of nucleic acids and proteins

(76) Inventors: Glauco R. Souza, Raleigh, NC (US); J. Houston Miller, Barnesville, MD (US)

Correspondence Address: CRAIG G. COCHENOUR, ESQ., BUCHANAN INGERSOLL, P.C., ONE OXFORD CENTRE, 20th FLOOR, 301 GRANT STREET, PITTSBURGH, PA, 15219, US

(21) Appl. No.: 10/436,621

(22) Filed: May 13, 2003

Related U.S. Application Data
(60) Provisional application No. 60/380,507, filed on May 14, 2002.

(51) Int. Cl. C12Q 1/68; G06F 19/00; G01N 33/48; G01N 33/50

(52) U.S. Cl. 435/6; 702/20

(57) ABSTRACT

This invention provides an apparatus and method that employs angle dependent light scattering combined with fractal dimension analysis of nanoparticle aggregates of gold and biopolymers, such as protein and nucleic acids, for detection and structural and functional characterization of unknown biopolymers. This is accomplished by detecting ADLS signal changes resulting from Au-biopolymer aggregate formation or from changes in fractal structure of Au-biopolymer aggregates as they specifically interact with other biopolymers. This invention describes an angle dependent light scattering apparatus that provides a sensitive, non-destructive, and dynamic measurement of the fractal dimension of Au-biopolymer aggregates, and provides a means for interpreting those measurements to allow identification of unknown nucleotides. A scattering cell is also provided.

### BENEFIT OF PRIOR PROVISIONAL APPLICATION

This utility patent application claims the benefit of co-pending U.S. Provisional Patent Application Serial No. 60/380,507, filed May 14, 2002, entitled “Fractal Dimension Analysis Of Nanoparticle Aggregates Using Angle Dependent Light Scattering For The Detection And Characterization Of Nucleic Acids And Proteins” having the same named applicants as inventors, namely, Glauco R. Souza and J. Houston Miller. The entire contents of U.S. Provisional Patent Application Serial No. 60/380,507 is incorporated by reference into this utility patent application.

### COMPUTER READABLE FORM AND SEQUENCE LISTING

Applicants state that the content of the sequence listing information recorded in computer readable form (CRF) as filed with this application is identical to the written paper sequence listing as filed with this application and contains no new matter as required by 37 CFR 1.821 (e-g) and 1.825 (b) and (d).

### BACKGROUND OF THE INVENTION

1. Field of the Invention

This invention relates to methods of providing structural and functional characterization of nanoparticle aggregates of gold and biopolymers (proteins and nucleic acids) by the use of fractal dimension analysis to interpret the results of angle-dependent light scattering. This invention also relates to the uses of a novel apparatus that allows the efficient implementation of the angle-dependent light scattering technique.

2. Description of the Background Art

I. Biopolymer Detection and Analysis

Recent technological advancements have increased the need for analytical methods that enable detection and characterization of biopolymers such as protein and DNA. See Cantor, C. R., et al., GENOMICS—The Science and Technology Behind the Human Genome Project, John Wiley & Sons: New York, 1999. Inexpensive devices and methods are available for biopolymer analysis; unfortunately, these traditional techniques, including PCR (polymerase chain reactions), immunoassays, gel electrophoresis, and membrane blots are often slow, require undue sample manipulation such as drying, and may generate undesirable waste. See Urdea, M. S., et al., Nucleic Acid Res. 1988, 16(11), 4937-4956. Other emerging techniques include DNA and protein arrays, electrospray mass spectrometry, matrix-assisted laser desorption, and ionization mass spectrometry. See Chee, M., et al., Science 1996, 274(5287), 610-614; Girault, S., et al., Anal. Chem. 1996, 68, 2122-2126; Griffin, T. J., et al., Nature Biotechnology 1997, 15, 1368-1372; Laken, S. J., et al., Nature Biotechnology 1998, 16, 1352-1356; Tang, K., Nucleic Acids Res. 1995, 32(16), 3126-3131. While use of these more recently-developed techniques circumvents some of the problems that arise from more traditional detection methods, the techniques are expensive and present a range of other difficulties. See Cantor, supra; Bowtell, D. D. L., Nature Genetics Suppl. 1999, 21, 25-32.

One promising technique for the detection of biopolymers involves the use of nanoparticle-oligonucleotide conjugates. See Mirkin, C. A., et al., Nature 1996, 382, 607-611; Elghanian, R., et al., Science 1997, 277 (5329), 1078-1081. Specifically, much attention has been paid to conjugates composed of gold (Au) nanoparticles to which thiol-modified oligonucleotides have been covalently attached. See FIG. 1. In the conjugate procedure, the thiol-modified oligonucleotides have been designed such that their terminus not bonded to the nanoparticles has a sequence complementary to the sequence of the biopolymer that they are meant to detect. Good examples of the conjugates may be found in U.S. Pat. Nos. 6,361,944 and 6,417,340, awarded to Mirkin, Letsinger, Mucic, Storhoff, and Elghanian When the nanoparticle-oligonucleotide conjugates are combined with their target complementary biopolymer under proper conditions, hybridization occurs. See Mirkin, C. A., et al., Nature 1996, 382, 607-611. As a result of this hybridization, an aggregate network of nanoparticle conjugates and biopolymers is formed. See Mirkin, supra; FIG. 2; FIG. 3. In the background art, the interaction is planned such that the existence of the aggregate causes a detectable color change that indicates the presence of the biopolymer. See U.S. Pat. Nos. 6,361,944 & 6,417,340. This technique is relatively inexpensive and thermally robust (stable), and it creates a minimum of undesirable waste. See Elghanian, supra.

Although use of the nanoparticle-oligonucleotide conjugates is effective for the detection of known biopolymers, utilization of the color change alone as a diagnostic tool fails to fully exploit the technique. More and better data is necessary and could be acquired, including the ability to identify the length and concentration of an unknown complementary biopolymer, and to examine the interactions of the complementary biopolymer with the nanoparticle conjugate dynamically in solution. The Au-oligonucleotide conjugates are particularly attractive for use as an analytic tool because they are inert, non-toxic, good light-scatterers, and easily acquired through manufacture or inexpensive purchase. See Dormemus, R. H., J. Chem. Phys. 1964, 40(8), 2389-2396; Frens, G., Nature Physical Science 1973, 241, 20-22; Oliveir, B. J, et al., J. Colloidal & Interface Science 1990, 134(1), 139-146; Oliveir, B. J., et al., Physical Review A 1990, 41(4), 2093-2100; Yguerabide, J., et al., Analytical Biochemistry 1998, 262, 157-176; Wilcoxin, J. P., et al., J. Chem. Phys. 1993, 98 (12), 9933-9950;Weisbecker, C. S., et al., Langmuir 1996, 12(16), 3763-3772; Takeuchi, Y., et al., J. Phys. Chem. B 1997, 101, 1322-1327.

The aim of this invention, then, is to provide a method and apparatus for using the nanoparticle-oligomer aggregate to provide useful data on, among other things, biopolymer length, concentration, and interaction. A collateral benefit of the invention will be an ability to increase available knowledge on the structure and creation of nanostructure forms.

II. Angle-Dependent Light Scattering

A powerful yet underutilized technique, angle-dependent light scattering detection (ADLS) is one way to non-invasively determine the distribution of particle sizes and morphologies in a gas, solid, or liquid. See Muller, R. H., et al., Particle and Surface Characterisation Methods, GmbH Scientific Publishers (1997); Stover, J. C., Optical Scattering Measurement & Analysis, The Society for Optical Engineering, Bellingham (1995); Harding, S. E., et al., Laser Light Scattering in Biochemistry, Redwood Press Ltd., England (1992); Young, A. T., Physics Today 1982, 35(1), 42-48; Bonczyk, P. A., Lagmuir 1991, 7, 1274-1280; Koylu, U. O., et al., Transactions of the ASME 1994, 116, 971-979, 152-159; Bryant, M. A., et al., J. Am. Chem. Soc. 1991, 113, 8284-8293. Until recently, ADLS was an impractical technology due to its high computational demands and its requirement that researchers use expensive, high quality light sources and detectors to receive consistent results. See Hunter, R. J., Introduction to Modern Colloid Science, Oxford Science Pubs., New York (1993); Brust, M., et al., J. Chem. Soc., Chem. Commun. 1994, 7, 801, 802; Chen, T. W., Applied Optics 1993, 32(36), 7568-7571; Evans, B. T. N., et al., Applied Optics 1990, 29(31), 4666-4670; Asano, S., et al., Applied Optics 1980, 19(6), 962-974; Asano, S., et al., Applied Optics 1975, 14(1), 29-49; Kucherlapti, Nature Genetics Supplement 1999, 21, 15-19. Due to its utility, ADLS was occasionally applied in the art despite its drawbacks; for instance, in U.S. Pat. No. 5,486,904, awarded to Horn, Lueddecke, Gierulski, Kroehl, and Lorencak, the technique was suggested as an alternative to the use of fluorescence for determination of the number and size of resin particles.

The general impracticality of ADLS has abated due to the ready availability and relatively low cost of powerful computers and quality optical equipment. While the decreased cost and increased availability of equipment necessary for ADLS have made use of the technique more feasible, traditional ADLS apparatus have suffered from data collection limitations that must be overcome before ADLS can become a truly effective analytic technique. Some ADLS systems have performed measurements only by collecting light at a single angle relative to the scattering cell. Although this method allows fast angular detection, it does not provide information on particle size unless used in conjunction with another, often destructive, technique, such as chromatography or gel filtration. See Gabriel, M. K., et al., FEBS Letters 1984, 175 (2), 419-421; Qian, R. L., et al., J. of ChromatographyA 1997, 787, 101-109; Korgel, B. A., et al., Biophysical Journal 1998, 74, 3264-3272; Machtle, W., Biophysical Journal 1999, 76, 1080-1091. Others have performed measurements using a rotating stage that moves a detector across different angles. This technique provides good resolution and allows differentiation of particle sizes without further activity; unfortunately, this type of apparatus operates with limited speed.

It would be desirable for one to be able to perform fast, efficient, ADLS using an apparatus capable of high angular resolution and differentiation of particle sizes. Given the suitability of the Au-oligonucleotide conjugate as a tool for analysis, such an apparatus would ideally be supplemented by a method for interpretation of ADLS/Au-oligonucleotide data that would provide information related to concentration and length of a target oligonucleotide.

Fractal dimension analysis is an effective method for calculating structural information. Fractal dimension may be defined as structures that are self-similar across scales. See Cohen, R. J., Benedek, G. B., Immunochemistry 1975, 12, 349-51. A basic example of fractal dimension analysis is seen in the “Koch snowflake,” shown at FIG. 4. Researchers in the fields of material and combustion sciences have used ADLS and fractal dimension analysis to study the structure and formation of soot and colloidal aggregates. Stauffer, D., Phys. Rep. 1979, 54, 1-74; Avnir, D., et al., Nature 1984, 308, 261-263; Koylu, U. O, et al., Combustion and Flame 1995, 100, 621-633. An aggregate of Au-biopolymer nanoparticles assembled by hybridization of Au-oligonucleotide probes to their target molecules is a good example of fractal structure.

Examination of the fractal dimension of a nanoparticle conjugate-target aggregate provides two useful pieces of information, as will be shown below. First, fractal dimension increases with increased concentration of the target molecule. This occurs because the increased target concentration causes the number of target molecules bonded to each nanoparticle conjugate to increase, resulting in a transition from string-like aggregates, with fractal dimension near 1, to more dense and compact structures with higher fractal dimension. See FIG. 5. Second, fractal dimension decreases with increasing target molecule length; longer targets allow greater solvation of the aggregate, resulting in less dense aggregate structures. See Martin, J. E., et al., J. Appl. Cryst. 1987, 20, 61-78. Fractal dimension has even greater utility when viewed in context of radius of gyration of the aggregate. Radius of gyration, a function of the mean primary particle radius and the separation between particles in a cluster, is also sensitive to the concentration of the target molecule; however, it is not as sensitive to changes in concentration as is fractal dimension.

Fractal theory postulates that the relationship between the number of primary particles (N) in a fractal aggregate and its radius of gyration (Rg) obeys the following relationship, where a is the mean primary particle radius, Df is fractal dimension, and kg is the fractal prefactor, a constant according to the following equation known by those skilled in the art:

$$N = kg \cdot {( \frac{Rg}{a} )}^{Df} \qquad ( Equation 1 )$$

The radius of gyration (Rg) is a function of a and 1, wherein 1 is the separation between individual particles in a cluster.

The angle-dependent light scattering signal is related to the fractal dimension of an aggregate according to the following relationship known by those skilled in the art:

N·I(q)≈kg·q(θ)^{-Df} (Equation 2)

where I(q) is the angle-dependent scattered light intensity, q(θ) is the scattering wavevector, which is a function of the scattering angle, θ, and the wavelength of the incident light, λ and Df is the fractal dimension of the scattering aggregate. This relationship is valid for small scattering angles, such that Rg⁻¹≦q(θ)≦a⁻¹; q(θ)=(4π/λ)·sin(θ/2). As will be appreciated by those skilled in the art, this relationship results from the interference between the scattered electromagnetic waves from the individual particles forming the fractal cluster.

### SUMMARY OF THE INVENTION

An angle-dependent light scattering apparatus is provided according to the present invention that is capable of non-destructive, sensitive, and dynamic measurement of a sample. The invention may be used to detect the light scattering signal changes in nanoparticle conjugates as they interact with and form aggregates with other biopolymers or small molecular weight molecules. These signal changes, when interpreted by fractal dimension analysis, provide information about the size and structure of the nanoparticle aggregates and the binding biopolymers with which they interact, allowing inference of the structural characteristics of the biopolymer and other information about the components of the solution as desired. It will be appreciated by those skilled in the art that the present invention is not limited to use with nanoparticle aggregates or biopolymers, and may be used with a variety of other molecules, including for example, but not limited to, small molecular weight pollutants or harmful biological agents.

One aspect of the instant invention is the novel configuration of the ADLS apparatus used to collect the scattering data. Unlike past apparatuses, which were limited as set forth above, the apparatus of the invention allows relatively fast measurements at high angular resolution through use of a rotating stage upon which are mounted both the sample cell and the detection devices.

In another embodiment of the present invention, the relationship shown in equations 1 and 2 above are employed to detect the interaction of a gold-biopolymer probe to DNA, proteins and small molecular weight molecules. Analysis of the scattering signal at small angles provides structure information about the gold-biopolymer clusters which allows for the extraction of quantitative information about the concentration and the structure of the gold-biopolymer aggregates. In an embodiment of the present invention, the number of the particles present in the gold-biopolymer cluster (N), equation 2 above, is dependent upon the concentration of the target DNA. In the present invention, the target nucleic acid concentration is determined by measuring the slope of the plot of I (q) versus the log of q. In another embodiment of the present invention, the length of DNA fragments are determined by taking advantage of the fractal dimension dependence on the ratio between the radius of gyration of a fractal cluster and its primary particle radius (a). Rg and Df are directly related to the distance between particles 1 and a. By using a range of nucleic acids with a known sequence and length (1), the present invention provides a calibration table that contains fractal dimension values corresponding to each 1 and a combination. The present invention includes a method for determining the fractal dimension for a target sample by employing light scattering measurements and the calibration table for determining the length of the target nucleic acid fragment.

In a more preferred embodiment of this invention, an angle dependent light scattering apparatus is provided comprising at least one electromagnetic beam, at least one polarizer for polarizing the electromagnetic beam, a lens and an iris for focusing the polarizing electromagnetic beam, a scattering cell for receiving the polarized electromagnetic beam, wherein the scattering cell is capable of containing a sample solution through which the polarized electromagnetic beam is passed to produce scattered light, at least one collection device for collecting the scattered light, a second polarizer for isolating the scattered light, a charge-coupled array detector (camera) for collecting the image of the polarized scattered light, and at least one computer (CPU) for coordinating the control of the operation of the angle dependent light scattering apparatus. The present invention provides an efficient apparatus for generation and collection of ADLS data and allows calculation of both the fractal dimension and radius of gyration of an Au-biopolymer/target molecule aggregate, a calibration table containing fractal dimension values corresponding to nucleic acids of known sequence and length for use in identifying unknown complementary molecules, and collecting ADLS information for an unknown sample, the data of which is then compared to the calibration table and the characteristics of the unknown sample revealed.

A scattering cell is provided comprising an outer reservoir and an inner reservoir for containing a sample solution. The scattering cell minimizes the refractive index changes between sample solution and glass and the refractive index difference between outside of the glass and ambient air.

In another embodiment of this invention, a method for determining at least one property of a test sample is provided.

### BRIEF DESCRIPTION OF THE DRAWINGS

Various other objects, features, and advantages of the invention will be readily apparent according to the following description exemplified by the drawings, which are shown by way of example only, wherein:

FIG. 1 shows an example of formation of an Au nanoparticle-oligonucleotide conjugate; the Au nanoparticle is attached to a biotin probe.

FIG. 2 shows an example of an aggregate of nanoparticle conjugates and their complementary biopolymers; Au-biotin probe conjugates are attached to the recombinant protein streptavidin.

FIG. 3 is a depiction of Au nanoparticle-DNA probe conjugate interaction with a target oligonucleotide to form an aggregate cluster.

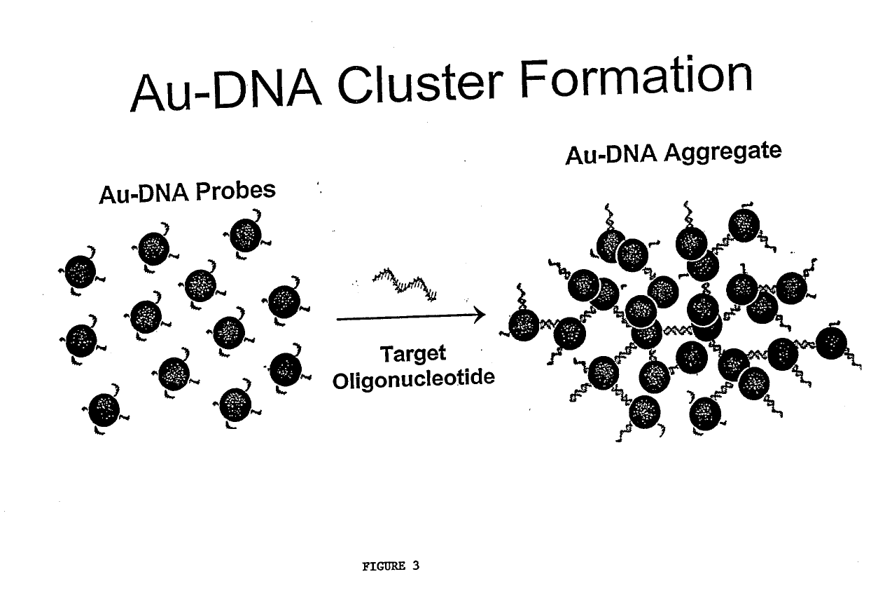

FIG. 4 displays the “Koch Snowflake,” an example of fractal structure.

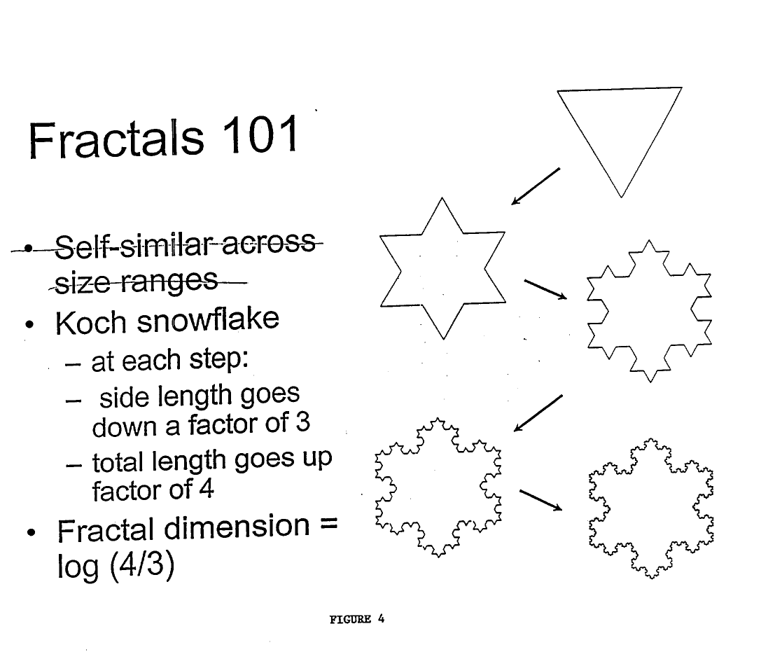

FIG. 5 illustrates the increase in fractal dimension corresponding with an increase in target oligonucleotide concentration.

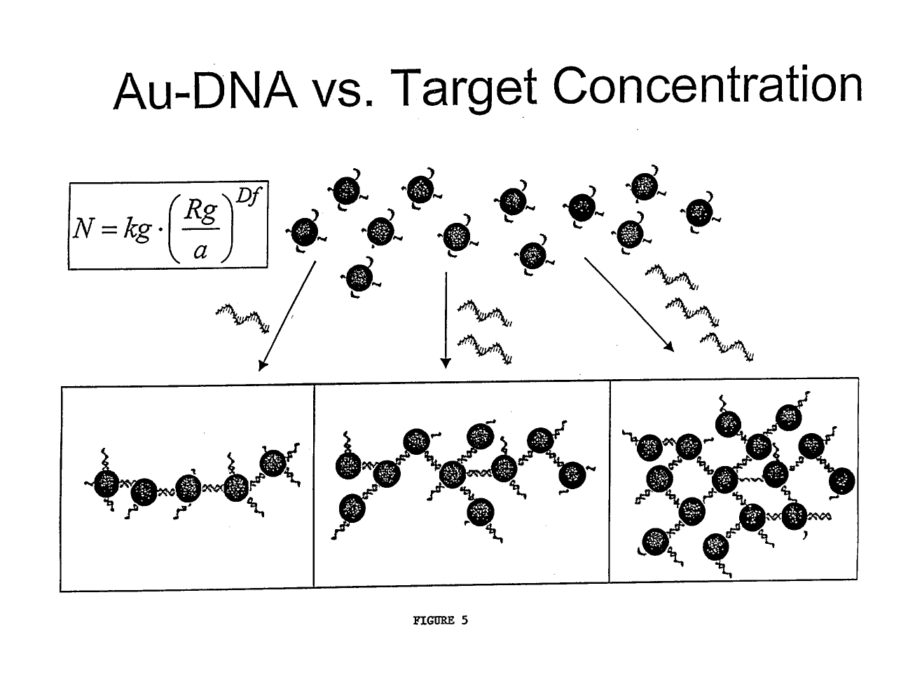

FIG. 6 shows angle dependent light scattering (ADLS) signal versus light-scattering angle for the hybridization of Au-DNA probes to different concentrations of 21 -base long oligonucleotide target. The ADLS signal comprises the raw scattering signal minus the background signal (scattering cell and hybridization buffer).

FIG. 7 is a Guinier analysis showing a plot of the natural logarithm of the scattering intensity versus the square of the wavevector. The different data sets are the angle-dependent scattering intensity (30°≦θ≦80°) for the four different concentrations of target oligonucleotide. The lines represent the linear fit for the low q region (θ≦70°) of each data set.

FIG. 8 is a fractal dimension analysis, in which ADLS data is plotted as log(Iᵥᵥ(q)/Iᵥᵥ(0)) vs. log(q), 30°≦θ≦80°. The linear regression analysis (lines) of each oligonucleotide target concentration was done for θ ranging from 30°≦θ≦60° (0.0034 less than or equal to q(θ) less than or equal to 0.010 nm⁻¹).

FIG. 9 is a schematic drawing of the preferred embodiment of the apparatus of the invention.

FIG. 10 is a graph of fractal dimension versus target oligonucleotide concentration for target oligonucleotides of lengths of twenty-one base pairs and thirty base pairs.

FIG. 11 shows the dioxin cycle wherein dioxin binds to aromatic hydrocarbon receptors (AhR), part of a heat shock protein (HSP90).

FIG. 12 shows a method of the present invention wherein Au-protein (HSP90) aggregates are employed for the detection of dioxin.

FIG. 13 shows a method of the present invention wherein Au-protein (Arnt) aggregates are employed for the detection of dioxin.

FIG. 14 is a schematic drawing of a preferred embodiment of the apparatus of the invention including employing an ellipsoidal mirror.

FIG. 15 is a schematic drawing of the ellipsoidal mirror and CCD array detector of the preferred embodiment of the apparatus of this invention.

FIG. 16 is a schematic drawing of an example of the dimensions of the ellipsoidal mirror of the apparatus of the present invention.

FIG. 17 is a schematic drawing showing image formation of the ellipsoidal mirror of the apparatus of the present invention.

FIG. 18 shows a graph of a second focal point position as a function of cell position calculated with Mathcad worksheet.

FIGS. 19A and B show angle dependent light scattering as a function of concentration of gold nanoparticle using the ellipsoidal mirror scattering apparatus of this invention illuminated with 532 nm and 660 nm of laser light, respectively.

FIGS. 20A and B show scattering signal as a function of the concentration of 100 nm Au using green laser angle calibration and red laser angle calibration, respectively.

FIG. 21 shows a drawing of the scattering cell of the angle dependent light scattering apparatus of the present invention.

FIG. 22 shows a drawing of a laser beam alignment through the center of the inner sample reservoir of the scattering cell of the apparatus of the present invention.

### DETAILED DESCRIPTION OF THE INVENTION

With reference to the remaining figures, the preferred embodiment of the invention will be described. The preferred embodiment will be directed primarily toward the use of the invention to analyze DNA using an Au-biopolymer aggregate, but those skilled in the art will appreciate that use of the apparatus and method is possible for any nanoparticle aggregate or target molecule, and that the apparatus and method is helpful for any instance in which use of ADLS is contemplated. Those skilled in the art will further understand that the described ADLS apparatus of the present invention may be made in a form that is more effectively portable than other detection devices of comparable utility set forth and known in the background art, and that the apparatus and method of the present invention allow dynamic examination of the protein interaction in their native environments.

As used herein, the term “small molecular weight molecules” includes those molecules having a mass of less than or about equal to two thousand (2,000) Daltons, and includes, for example but not limited to, peptides, protein co-factors and environmental toxins.

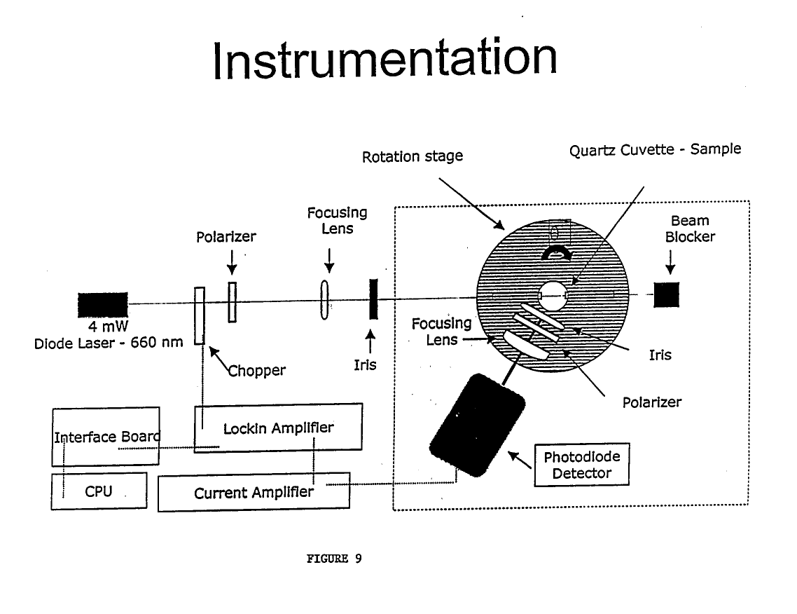

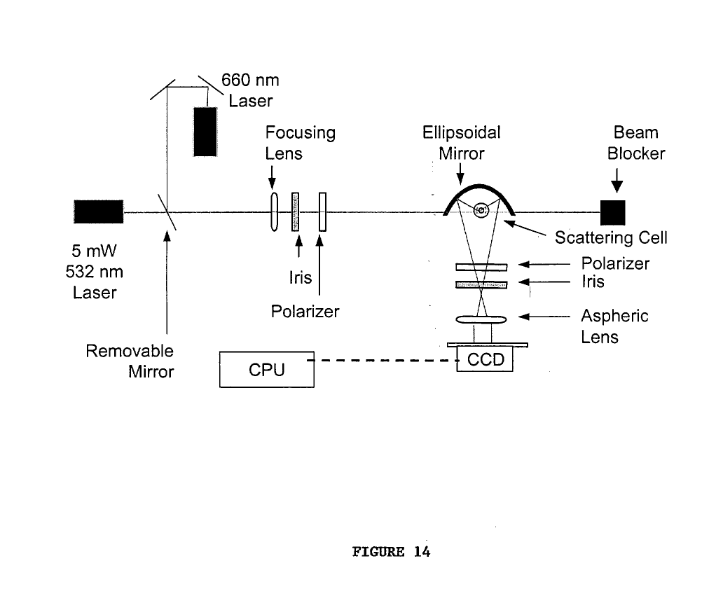

Schematic diagrams of preferred embodiments of the apparatus of the present invention are shown in FIGS. 9 and 14. In a preferred embodiment of this invention an angle dependent light scattering apparatus is provided comprising at least one electromagnetic beam, at least one polarizer for polarizing the electromagnetic beam, a lens and an iris for focusing the polarizing the electromagnetic beam, a scattering cell for receiving the polarized electromagnetic beam, wherein the scattering cell is capable of containing a sample solution through which the polarized electromagnetic beam is passed to produce scattered light, at least one collection device for collecting and collimating the scattered light, a second polarizer for isolating the collimated and scattered light, another lens (a second lens) for focusing the collimated and scattered light onto a plane into at least one analysis device for analyzing the data, and at least one computer for coordinating the control of the operation of the angle dependent light scattering apparatus, wherein at least the scattering cell and the collection device are on a stage capable of rotation. This preferred embodiment of the apparatus of the present invention is set forth in FIG. 9, wherein a a photodiode laser head is comprised of a 4 mW Fabry-Perot diode laser, with an output wavelength of 660 nm, mounted in a thermoelectric cooled laser mount, which contains a collimation lens alignment mechanism. The 660 nm laser wavelength was chosen because the Au nanoparticle maximum cross section of scattering is approximately at 650 nm, and 660 nm photodiode lasers are inexpensive, powerful, and readily available. The collimated laser beam first passes through a mechanical chopper (1 KHz frequency), which triggers a lock-in amplifier for phase-sensitive detection. The collimated laser beam then passes thorough a perpendicular polarizer. Next, the polarized light passes through a focusing lens and an iris, which focus the polarized laser beam into the center of a 1 cm diameter quartz sample cell (scattering cell) through a 40 cm focal length lens. Excess light is prevented from escaping the unit by a beam blocker. Optionally, a thermoelectric device (not shown in FIG. 9) is attached to the scattering cell and is in contact with the sample solution for controlling the solution temperature. The scattered light is collected by a 1″ aspheric lens with a focal length of 2.1 cm (centimeter) placed 2.1 cm from the center of the scattering cell. This configuration geometry allows the scattered light to be collimated and it also gives the aspheric lens a field view angle of about 90 degrees. The field view angle is defined by the angle formed between the center of the edges of the aspheric lens. Further, by centering the two collimating lenses at about a 45 degree angle (θ) or at about a 135 degree angle (θ) relative to the incident beam, we detect approximately 180 degrees of the angle dependent scattered light intensity (scattering profile). After the scattered light is collimated, it passes through a polarizer which isolates the scattered light with the same polarization as the incident light. The polarizer sits in a rotation mount. The polarizer isolates perpendicularly polarized scattered light and allows that perpendicularly polarized light to pass to a photodiode detector. All of the light collection components are on the rotation stage. The signal generated by the photodiode detector is sent to a current amplifier, then transferred in turn to the lock-in amplifier, an interface board, and a central computer.

The central computer controls all motorized parts, electronic instrumentation, data storage and data analysis. The central computer controls all motorized parts, electronic instrumentation, data storage, and data analysis. A computer program continuously monitors the laser power so that any scattering signal changes caused by laser power fluctuations can be corrected. Laser power and photodiode integration time are also automatically adjusted according to charge saturation levels of the photodiode detector. The thermoelectric couple is the primary trigger for measurements, and data is collected only at user-defined temperature settings. Every scattering profile is stored with all accompanying scan conditions, such as temperature, polarization of light, and rotation stage angular position. In the preferred embodiment, the data is analyzed using a Mathcad worksheet (commercially available from Mathsoft Engineering & Education, Cambridge, Mass.) that manipulates and compares the scattering profile for each polarization of light to the expected scattering profile for each particle size in solution. The preferred embodiment is designed with the recognition that an inherent challenge in all light scattering measurements is to reduce the background generated by stray light from reflection, diffraction, and scattering from optical components. See B. Chu, Laser Light Scattering, Academic Press, New York, 1974. Stray light generally results from imperfections in the glass wall of the scattering cell and from scattering cell reflection and diffraction due to changes in the refractive index at the scattering cell's air/glass and glass/liquid interfaces. In one preferred embodiment of this invention the problem of stray light due to imperfections in the scattering cell is minimized by use of a low scattering glass, as known by those skilled in the art, such as for example, glass having a refractive index of about 1.52, including but not limited to employing BK7 glass as the scattering cell material.

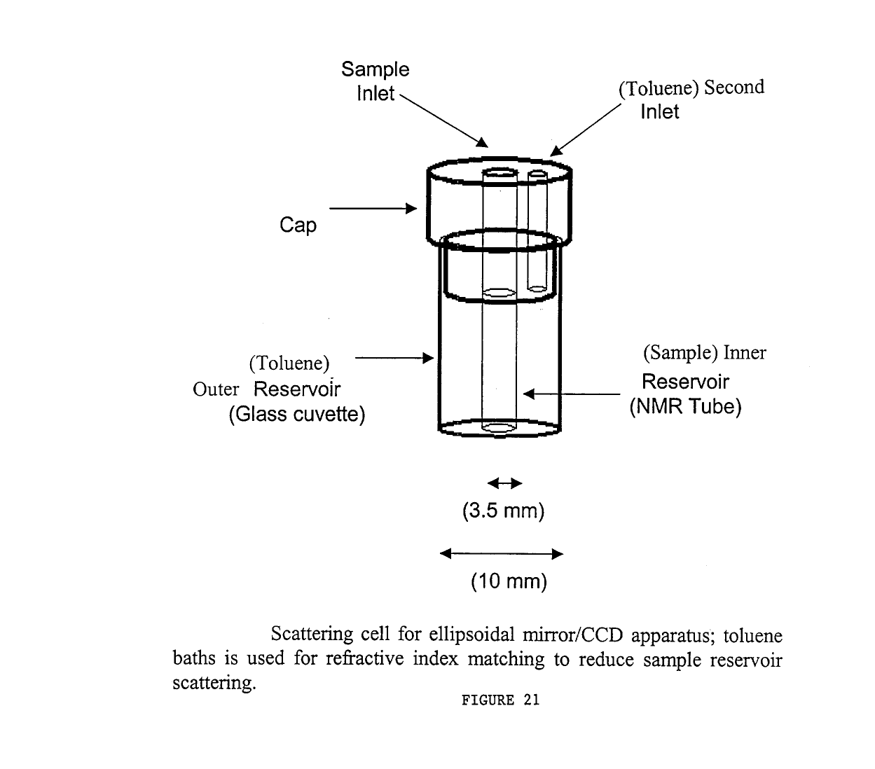

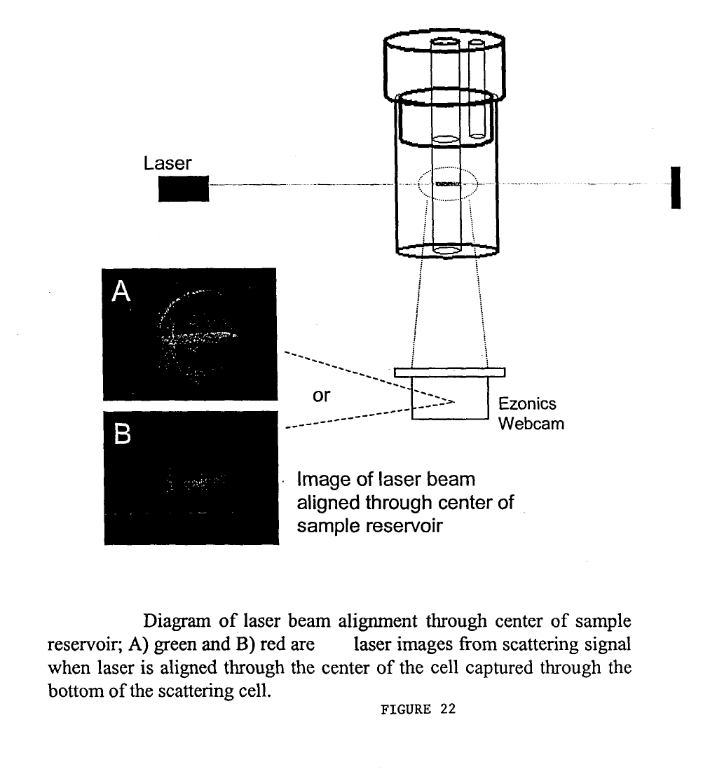

In a more preferred embodiment of the present invention, a scattering cell is provided as set forth in FIG. 21 which comprises an outer reservoir including a sealed first end and a second end and a body disposed between the first end and the second end, wherein the second end has a removable cap that is in sealing engagement with the second end, and an inner sample reservoir that is located within the outer reservoir, and wherein the inner sample reservoir has a first sealed end, a second end, and a body disposed between the first and the second end of the inner sample reservoir. The second end of the inner sample reservoir is in juxtaposition to the removable cap of the outer reservoir. The inner sample reservoir may have for example a tubular shape and may be such as for example but not limited to a NMR tube. The outer reservoir may have for example a tubular shape and be for example a glass cuvette. Optionally, the scattering cell removable cap has a sample inlet that is in communication with the inner sample reservoir. Optionally, the scattering cell removable cap has a second inlet that is in communication with the outer reservoir. The design of the scattering cell of this invention minimizes the refractive index changes between the sample solution and the glass and also the refractive index difference between outside of the glass and ambient air. In this most preferred embodiment of the scattering cell of the present invention, a tubular inner sample reservoir is provided that is immersed in a tubular outer reservoir wherein the tubular outer reservoir contains toluene. FIG. 21 shows for example that the diameter of the inner sample reservoir is about 3.5 millimeter (mm) and that the diameter of the outer reservoir is about 10 mm. The problem of the change in refractive index is corrected through use of this more preferred scattering cell of this invention. FIG. 22 shows a diagram of a laser beam aligned through the center of the inner sample reservoir of the scattering cell of this invention. FIG. 22 A shows green laser images and FIG. 22 B shows red laser images from scattering signal captured through the bottom of the scattering cell of this invention.

The data having been collected, it is compared with tabulated data using the fractal dimension method to determine the length and configuration of the sample. In the preferred embodiment, the sample is comprised of Au nanoparticles combined with thiol-modified oligonucleotides, as well as a target oligonucleotide. The Au nanoparticle-oligonucleotidc conjugates in the sample are hybridized with the target nucleotides to form aggregates.

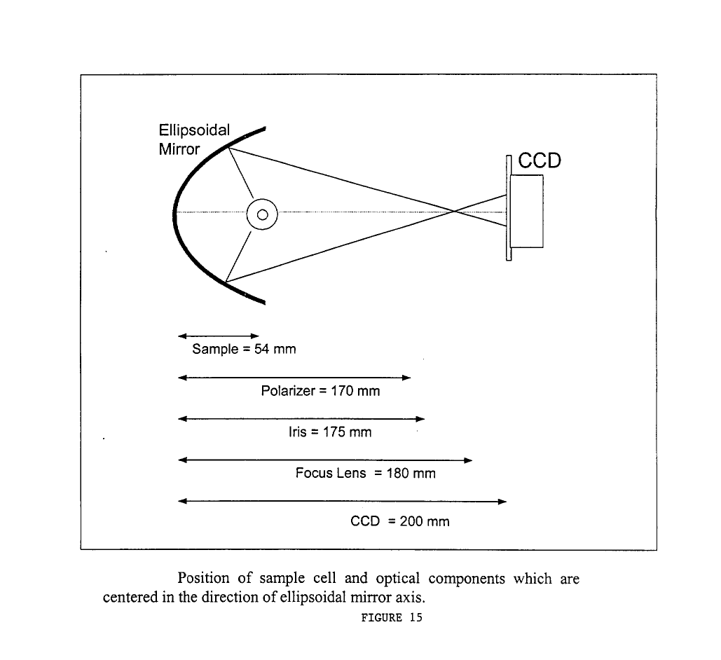

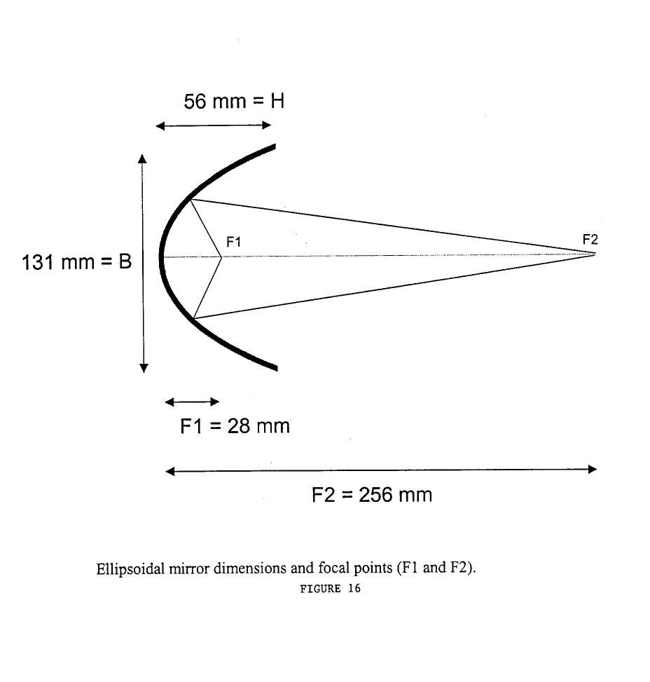

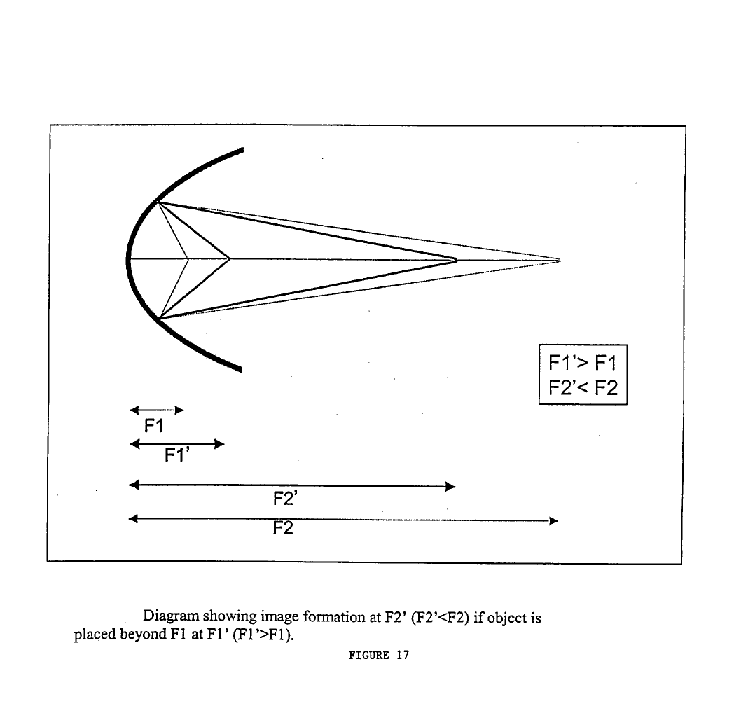

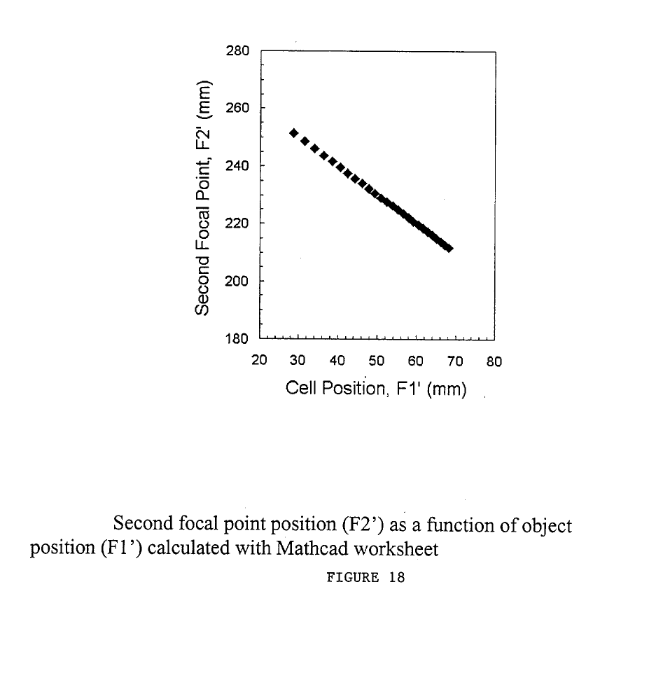

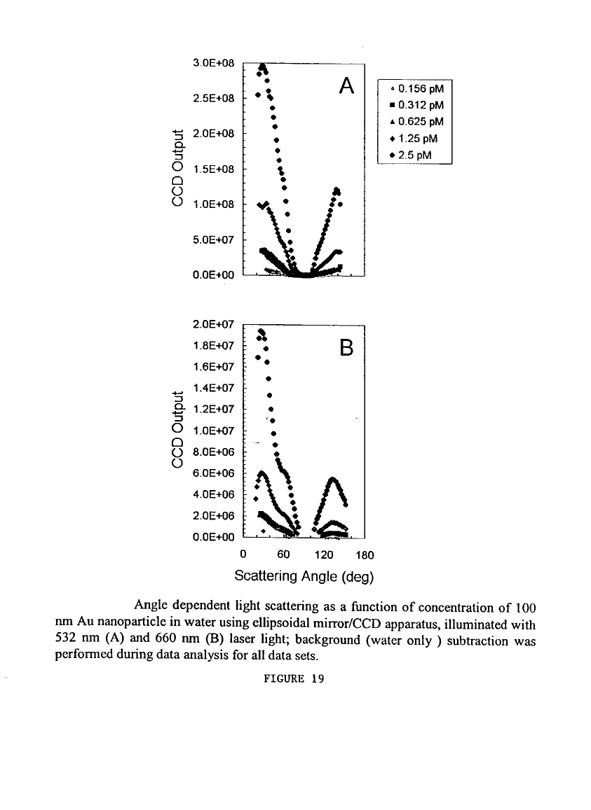

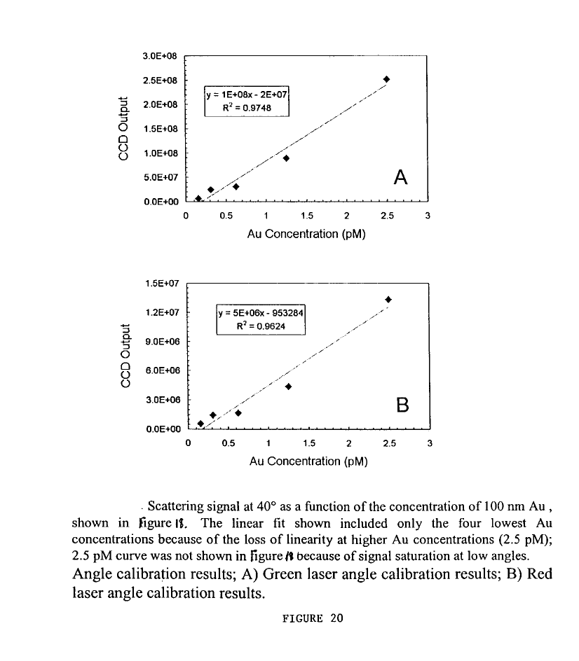

In a more preferred embodiment of this invention an angle dependent light scattering apparatus is provided comprising at least one electromagnetic beam, at least one polarizer for polarizing the electromagnetic beam, a lens and an iris for focusing the polarizing electromagnetic beam, a scattering cell for receiving the polarizing electromagnetic beam, wherein the scattering cell is capable of containing a sample solution through which the polarized electromagnetic beam is passed to produce scattered light, at least one collection device for collecting the scattered light, a second polarizer for isolating the scattered light, a charge-coupled device (CCD) array detector (camera) for collecting the image of the polarized scattered light, and at least one computer for coordinating the control of the operation of the angle dependent light scattering apparatus. In this more preferred embodiment of the angle dependent light scattering apparatus of this invention each light collecting device is stationary. In a most preferred embodiment of this invention, the collection device for collecting the scattered light is an ellipsoid mirror. FIG. 14 shows this more preferred embodiment of the apparatus of the present invention, as described herein, further including an ellipsoidal mirror and a cylindrical lens focuses the collimated light horizontally onto the scattering plane into the CCD array detector (identified in FIG. 14 as “CCD”) preferably having a 1″ CCD chip (1024×256 pixels). The angular resolution is defined by the width of the CCD pixels (26 μm/pixel). This optical configuration, combining an aspheric lens, a cylindrical lens and the CCD array detector, can detect approximately 90 degree angle scattering profile in one single measurement with an angle resolution of approximately 0.2 degree per pixel. FIG. 15 shows the position of the scattering cell having the sample solution and the optical components of the present invention which are centered in the direction of the ellipsoidal mirror axis. FIG. 16 shows an example of the ellipsoidal mirror's dimensions and focal points, Fl and F2, respectively. FIG. 17 sets forth a diagram showing image formation at F2′ (F2′<F2) if object is placed beyond Fl at Fl′ (F1′>F1). FIG. 18 shows a second focal point position F2′ as a function of object position F1′ calculated using the Mathcad worksheet. FIG. 19 sets forth an example of the angle dependent light scattering as a function of concentration of 100 nm (nanometer) Au (gold) nanoparticle in water using the more preferred embodiment of the apparatus of this invention, illuminated with 532 =m laser light (FIG. 19 A) and 660 nm laser light (FIG. 19 B). FIG. 20 shows the scattering signal at 40 degrees as a function of the concentration of 100 nm Au as shown in FIG. 18. FIG. 18 A shows the green laser angle calibration results and FIG. 20 B shows the red laser angle calibration results.

### EXAMPLES

Au-DNA Studies

In a preferred embodiment, the present invention provides a method employing ADLS/FD (angle dependent light scattering and fractal dimension) to the in-situ detection of specific nucleic acid sequences and study of Au-DNA cluster fractal dimension in a liquid sample. The coupling of ADLS/FD with Au-DNA probes of this invention can quantitatively determine sequence, concentration and length of nucleic acids. The method of the present invention offers affordable, rapid and sensitive detection, which is ideal for high throughput analysis of nucleic acids such as for forensic detection, for clinical diagnostics, such as for example but not limited to detection of cancer, detection of genetic related diseases, detection of pathogenic microorganisms and any other nucleic acid detection application, and for genetic therapy in the treatment of diseases such as for example but not limited to cancer, genetic diseases, and for understanding and slowing the aging process, and for building nano-structures. The method of this invention can be further implemented for in-situ detection of PCR products, where one could detect the sequence, concentration and length of the hybridizing oligonucleotide strands as the nucleic acid amplification takes place.

The angle-dependent light scattering signal and fractal dimension behavior of Au-DNA probes of this invention and the time dependent and static studies of ADLS signal from Au-DNA probes and clusters as a function of: polarization of light; Au nanoparticle size and Au-DNA probe concentration; Au-DNA probe synthesis and hybridization conditions, such as presence of salt, organic solvent and mercaptohexanol; concentration changes of one Au nanoparticle size in a mixture containing various Au nanoparticle sizes; simultaneous concentration changes of two or more sizes in a mixture containing various Au nanoparticle sizes; Au-DNA probes change in concentration as it hybridizes to the target DNA; target DNA concentration and reversibility of hybridization process; fractal dimension as function of Au nanoparticle size and concentration; fractal dimension as a function of thiol-modified length, concentration, and annealing temperature; fractal dimension as a function of target DNA concentration, length, and annealing temperature; fractal dimension as a function of solution conditions, such as, ionic strength, buffer, and pH; and reverse aggregation study where Au-DNA probes are clustered together and then a complementary target is introduced reversing the aggregation, have been performed. Atomic force microscropy (AFM), scanning tunneling microscopy (STM), and Uv-vis spectroscopy as complementary techniques were used to confirm Au-DNA cluster formation and DNA hybridization.

Thermodynamic study of the hybridization of the Au-DNA probes as they form the Au-DNA clusters was also performed for accurately predicting DNA hybridization (annealing) temperatures as a function of Au-DNA probe sequence and length under optimized experimental conditions. There are many empirical and theoretical models for determining DNA annealing temperatures (Cantor and Cassandra, 1999; Machtle, 1999; Patersheim and Turner, 1983). Unfortunately, DNA annealing depends not only on temperature and base sequence, but it also depends on solution conditions, such as ionic strength, metal content and presence of organic solvents. By accurately predicting the annealing temperature of a Au-DNA probe sequence, we can control the temperature of the sample and selectively detect the hybridization of each Au-DNA probe in the sample.

Chemistry of Oligonucleotide Binding to Au.

The Au nanoparticles, thiol modified oligonucleotides and DNA targets are commercially available. The Au nanoparticles range from about 1 to greater than about 100 nanometers in diameter and preferably are from about 50 nm (nanometers) to about 300 nm in diameter. The thiol modified oligonucleotide (HS-DNA primer) comprise artificially synthesized oligonucleotides readily available in any specific sequence with a preferred maximum length of about 80 bases. More preferably, oligonucleotides no longer than about 40 bases long were employed. The DNA targets comprise artificially synthesized oligonucleotides of specific sequences (DNA primers) and of DNA PCR products of known length. The Au-DNA probes were prepared by covalently binding the HS-DNA primer to Au nanoparticles of specific sizes through the thiol moiety. The chemistry of adsorption of thiols on to Au has been extensively studied (Weisbecker et al., 1996; Whitesides et al., 1993; Barndad, 1998; Bryant and Pemberton, 1991). The preferred mechanism of this chemisorption process is as follows (Weisbecker et al., 1996):

(Au nanoparticle)⁻+HS-DNA→(Au nanoparticle)-S-DNA+½H₂

The extent of HS-DNA primer bound to the Au nanoparticle is dependent on solution conditions, such as salt concentration and presence of organic solvents (Storhoff and Mirkin, 1999; Peterlinz et al., 1997; Heme and Tarloy, 1998; Lewis and Tarlov, 1995). The initial experimental conditions that are used are those described in the literature (Elghanian et al., 1997; Herne and Tarlov, 1998). Even though the adsorption of thiol modified DNA to gold nanoparticles reduces its non-specific aggregation (Elghanian et al., 1997), our scattering measurements indicate that the DNA modified Au nanoparticles are still susceptible to non-specific aggregation due to solution conditions, such as ionic strength and pH.

Protein Characterization

We describe the initial studies for validating fractal dimension analysis combined with angle-dependent light scattering (ADLS/FD) and gold (Au) nanoparticles to detect and characterize proteins and protein nanoparticle aggregates. The general approach for this methodology is to modify gold nanoparticles with a ligand moiety, usually a cofactor or a protein, that specifically interacts with a target protein, where the interaction between the moiety and the target protein induces the assembly of the gold nanoparticles into a Au-protein aggregate.

Au nanoparticles are modified with a ligand, a small molecular weight molecule, that interacts with a target protein that has two, three or four ligand binding sites. The Au-protein aggregation results from Au-ligand interaction with the target protein. Fractal dimension (Df) increases as the number of binding sites increase. The increase in Df is followed by a transition from string like aggregates (lower values of Df) to denser, more compact structures with higher fractal dimension. The fractal dimension (Df) dependence of the Au-DNA aggregates is a function of target DNA concentration and length. With the assumption that Au-protein aggregates have fractal structure, the structural differences of Au-Protein aggregates should be a function of the characteristics of the protein linking the Au nanoparticles, which implies that the fractal dimension is sensitive to the size, shape and position of the binding sites of the linking protein.

One of the innovative aspects of the work described in this chapter is the possibility of characterizing protein and protein-nanoparticle structures in the protein native environment. The main techniques for structure characterization of proteins are X-Ray crystallography and NMR (Chreighton 1993; Garrett and Grisham 1995; Pennington and Dunn 2001). X-Ray crystallography is a mature technique that can reveal detailed three-dimensional information about the structure of a protein or a nucleic acid. NMR can provide localized information about the structure of a protein, such as the topology of a polypeptide chain in solution (Chreighton 1993; Pennington and Dunn 2001). The structure provided by NMR is not as detailed and accurate as X-Ray crystallography, but it allows measurements to be made in solution. The great challenge of using X-Ray crystallography is that the process of growing protein crystals that are suitable for X-Ray crystallography is difficult and tedious. Many of the persons skilled in the art consider crystallization an art more than a science (Stryer 1988). Generally crystallography data is accurate and provides detailed structure of a crystallized protein, but this structure may not represent its native structure. Crystallizing agents that are often added for aiding the crystallization process and the crystallization itself can induce conformational changes in the protein. In the case of NMR, some of the drawbacks are the limited range of structure information and the complexity of interpreting the results. X-Ray crystallography and NMR require high-energy sources and complex instrumentation. The method of the present invention is a good complement for both crystallography and NMR. It provides preliminary structural information about number of binding sites which generally translates into the number of homologous units in a protein, and also information about the position of these binding sites. This information aids in interpreting both crystallography and NMR. The capability of performing real time measurements using solution conditions which mimic the native protein environment, is a great asset when characterizing proteins and other biopolymers.

As far as the characterization of Au-Biopolymer aggregates, microscopy techniques are the most widely used technique (Mirkin, Letsinger et al. 1996; Muller and Mehnert 1997). However, microscopy presents limitations when characterizing these aggregates. First, most microscopy measurements are ex-situ, therefore they are inadequate for real time detection of Au-Biopolymer cluster formation. Second, the information obtained from microscopy methodology can be compromised because most of these techniques require special handling of the sample, such as drying the sample on a surface. The process of drying can impose structure changes that are not characteristic of the actual cluster formation. In addition, microscopy images provide mostly 2D information about the structure of these clusters. X-ray scattering and dynamic light scattering are also standard tools for probing the structure of clusters. However, X-ray scattering and dynamic light scattering present clear drawbacks in comparison to the ADLS/FD technique. X-ray scattering requires high energy photons which are not routinely available. The main limitation of dynamic light scattering is that traditionally this instrumentation uses single angle detection, which dramatically impairs the dynamic range of this technique. The protein system presented herein is the streptavidin-biotin complex. The strong biotin-streptavidin interaction and the four available binding sites allow streptavidin to be used as a cross-linker between biotin modified gold nanoparticles (Au-Biotin probe), forming a fractal assembly of Au and protein (Au-protein aggregate) We studied Df and Rg of Au-biotin-streptavidin aggregation under specific protein and experimental conditions. We detected the binding of biotin to streptavidin (monomer) and to a streptavidin-streptavidin complex (complex) by monitoring changes in the angle-dependent scattering signal produced by the Au-Biotin probe as it binds to either the streptavidin monomer or complex. We probed the fractal dimension of the Au-protein aggregates from the analysis of ADLS measurements, with the assumption that fractal dimension differences between of Au-protein aggregates assembled with either monomer or complex would indicate structural differences between the monomer and complex.

We synthesized streptavidin-streptavidin complex (mixture of monomers, dimers, trimers and larger complexes) using protein conjugation chemistry (Avrameas 1969; Wong 1993), so we have two types of protein samples, monomer and complex sample, which have the same chemical function but different size, shape, number and position of binding sites. By using this streptavidin-complex we studied Df and Rg as a function of size, number/position of binding sites. Au-Biotin probes were made by labeling disulfide-modified biotin with gold nanoparticles of specific sizes (chemistry detail given in experiment description).

We show that where we use Au nanoparticles modified with biotin and streptavidin, the specific interaction between streptavidin and biotin induces the aggregation of the Au-biotin nanoparticles. We also present an experiment where fractal dimension analysis and light scattering was used to detect the binding of commercially available immunoglobulin (antibody) modified gold nanoparticle to its target antigen.

Thiol-Biotin Conjugation

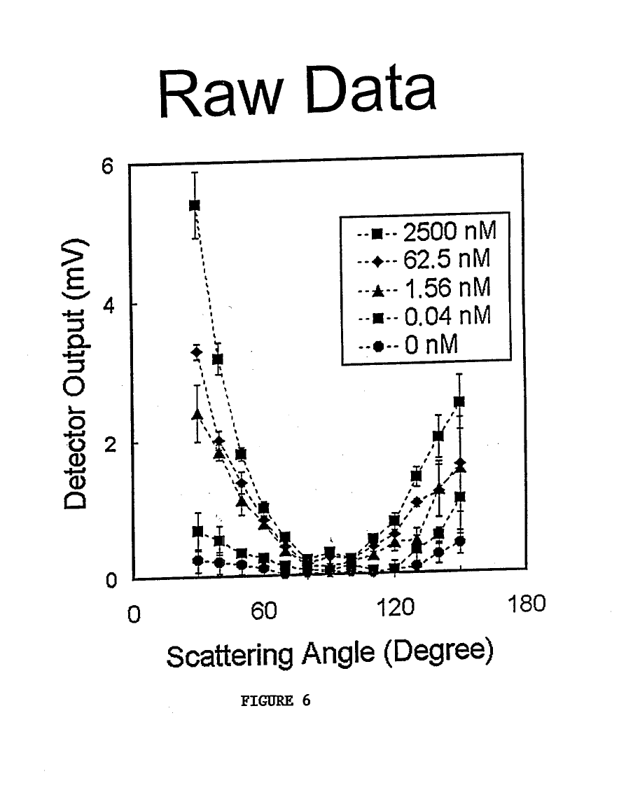

The approach taken to functionalize the Au nanoparticles was to use disulfide moiety to anchor biotin to the surface of the Au nanoparticles. The first step was to chemically modify biotin with a disulfide-containing molecule. The molecule of choice was 1-cystine (cystine). Cystine is an amino acid derived from two cysteine bridged through a disulfide bond, as shown in FIG. 6. The choice of using the disulfide form instead of plain cysteine or another form amine-thiol containing molecule, such as meracaptoethalamine, was because it has been reported that gold self-assembled monolayers (SAM) originated from di-n-alkyl disulfides (RSSR) are more stable than those those from alkanethiols (RSH) (Nuzzo, Zegarski et al. 1987; Ulman, Evans et al. 1992; Ulman 1996; Weisbecker, Merritt et al. 1996; Gronbeck, Curioni et al. 2000).

The coupling chemistry used NHS ester modified biotin to generate the disulfide modified biotin. The NHS ester group reacts with the deprotonated form of the primary amine. The amine reacts with the NHS ester by nucleophilic attack, which forms a stable amide linkage and N-hydroxysuccinimide is released as the by-product of the reaction (Wong 1993; Hermanson 1996). NHS ester cross-linkers are routinely used for modifying proteins and protein cofactors, such as biotin. Hydrolysis of the NHS ester is a major competing reaction in aqueous solution, and the rate of hydrolysis increases with increasing pH.

Biotin modified with NHS ester and other reactive cross-linkers is readily available from a variety of sources, our choice was the sulfo-NHS-LC-LC-biotin from Pierce. We chose sulfo-NHS form because of its better solubility in aqueous environment than the original NHS form, which usually has to be dissolved in dimethyl sulfoxide (DMSO), dimethyl formamide (DMF). Although DMSO and DMF can retard the rate of hydrolysis of the very reactive NHS group, they are not environmentally friendly and extra purification steps are required for their removal since they can denature proteins. The LC-LC region of this molecule adds extra length of the modified moiety, which can aid in minimizing hindrance effects that could exist because of the close proximity between the Au nanoparticle and the streptavidin binding site.

The disulfide carrying molecule, cystine, contain two primary amines available to react with NHS ester, which we react the sulfo-NHS biotin with the cystine in pH greater than 9.1. The high pH is required to assure that mostly the deprotonated primary amines are present in solution to assure efficient reaction. The pKa for the primary amine of cystine is approximately 9.1 (Chreighton 1993), which assures the abundance of deprotonated primary amines. The buffer of choice was 20 mM borate buffer at pH 9.35 (30% 20 mM boric acid and 70% 20 mM sodium borate—borate buffer), where borate buffer is an amine free buffer which also has preservative properties that assures a long shelf life for these solutions.

The actual procedure for the modification of biotin comprised first preparing a 0.25 mM cystine solution in borate buffer (cystine solution); after cystine is fully solubilized, the lyophilized sulfo-NHS-biotin is weighed in a clean small volume and capped tube (15 ml). Because of the small mass of the sulfo-NHS-biotin being measured, we first determine the weight of sulfo-NHS-biotin in the flask and then determine the volume of cystine solution to be added. The volume of cystine to be added was determined so that there is a 2× molar excess of sulfo-NHS-biotin, for example: if there was 12 mg of sulfo-NHS-biotin (17.9×10⁻⁶ moles, MW 669.75) in the flask, we added 12.5 ml of 0.45 mM cystine to the 12 mg of sulfo-NHS-biotin. After combining the cystine solution and the sulfo-NHS-biotin, this solution was incubated for 1 hour at room temperature. After one hour 10 μL of 0.5 M glycine in borate buffer solution was added to stop the reaction, where the primary amine of glycine reacts with the remaining unreacted sulfo-NHS-biotin. We label the final solution as 450 μM 1-cystine-biotin in 20 mM borate buffer (cyss-biotin).

Gold-Biotin Coupling

The covalent linkage between the Au nanoparticle and the disulfide-modified biotin (1-cystine-biotin or cyss-biotin) was done through the disulfide bond of the cystine and the Au nanoparticle. The preferred mechanism, which has been supported by experimental data and theoretical calculations (Nuzzo, Fusco et al.; Nuzzo, Zegarski et al. 1987; Porter, Bright et al. 1987; Bain, Biebuyck et al. 1989; Walczak, Chung et al. 1991; Ulman, Evans et al. 1992; Gronbeck, Curioni et al. 2000), is the one where the disulfide bond is broken close to the surface with the generation of two thiolates (RS-) which then adsorbs onto Au (Ulman 1996):

R—S—S—R+Au^{o}ₙ→R—S⁻Au⁺·Au^{o}ₙ

Existing literature shows that the rates of adsorption of n-alkyl-disulfides is indistinguishable from alkanethiols, but the rate of replacement for thiols is much faster than for disulfides (Biebuyck and Whitesides 1994; Ulman 1996; Gronbeck, Curioni et al. 2000). It also has been suggested that the estimated adsorption energy for dialkyl disulfides is twice as favorable as the adsorption energy for thiols (Schlenoff, Li et al. 1995; Ulman 1996). Determining the exact mechanism of adsorption of thiols and disulfides on gold nanoparticles is not a easy task (Gronbeck, Curioni et al. 2000), which explains different opinions of those skilled in the art on the exact mechanism of thiol and disulfide chemisorption on gold (Ulman, Evans et al. 1992; Whitesides, Ferguson et al. 1993; Ulman 1996; Weisbecker, Merritt et al. 1996). The theoretical coverage of a SAM on Au is approximately 0.77 nmol/cm² (Weisbecker, Merritt et al. 1996).

The procedure for the attachment of biotin to the Au nanoparticles was done by first mixing cyss-biotin with Au solution (40 nm or 100 nm), and allowing this solution to incubate for at least 24 hours. When designing the procedure for attaching the cyss-biotin, we first performed a study to probe the behavior of the 40 nm and 100 nm Au nanoparitcle in the presence of different concentrations of disulfide bond of cystine. The cystine concentration ranged from 0.8 mM to 12.5 mM and the Au concentrations were 34 pM and 3 pM of 40 nm and 100 nm Au, respectively. The 100 nm Au nanoparticles showed nearly no changes in their spectra as a function of concentration. We detected a small increase in absorption intensity at 564 nm, likely resulting from surface plasmon resonance changes resulting from adsorption of cystine (Weisbecker, Merritt et al. 1996). The absorption around 260 nm results from cystine absorption through the disulfide group, which absorption increases as a function of cystine concentration. We did not attempt to use higher concentrations of cystine because of cystine's poor solubility at higher concentrations than 12.5 mM. In contrast to 100 nm Au spectra that nearly stayed unchanged, the 40 nm Au particles showed a red shift for their absorption for cystine concentrations above 1.56 mM. The longer wavelength absorption results from Au aggregation, which induces a shift in the Au nanoparticle surface plasmon absorption wavelength.

When a thiol or disulfide containing molecule adsorbs onto the surface of a Au nanoparticle, the overall surface charge of the Au nanoparticle changes. This charge displacement can induce an aggregation of the Au nanopartildles, as observed for the cystine experiment above. Often, Au nanoparticles carry a overall negative charge resulting from either citrate groups present on its surfaces resulting from the citrate reduction process of its synthesis, or from hydroxy and chloride groups present in solution which weakly adsorb onto Au nanoparticles (Weisbecker, Merritt et al. 1996); because of this negative net charge, the Au nanoparticles can repel each other. Once these charges are displaced during the adsorption process, such as for thiols or disulfides, these charges can be displaced, changing the repulsive nature of the nanoparticles. This can allow these particles to approach each other, and once their separation is small enough (∝d⁻⁶, d=separation between nanoparticles), colloidal forces, such as van der Walls and hydrophobic interactions, induce aggregation. For Au nanoparticles, the aggregation process can be detected by monitoring the red shift in the plasmon resonance absorption wavelength when aggregation takes place (Whitesides, Ferguson et al. 1993; Weisbecker, Merritt et al. 1996; Reynolds, Mirkin et al. 2000).

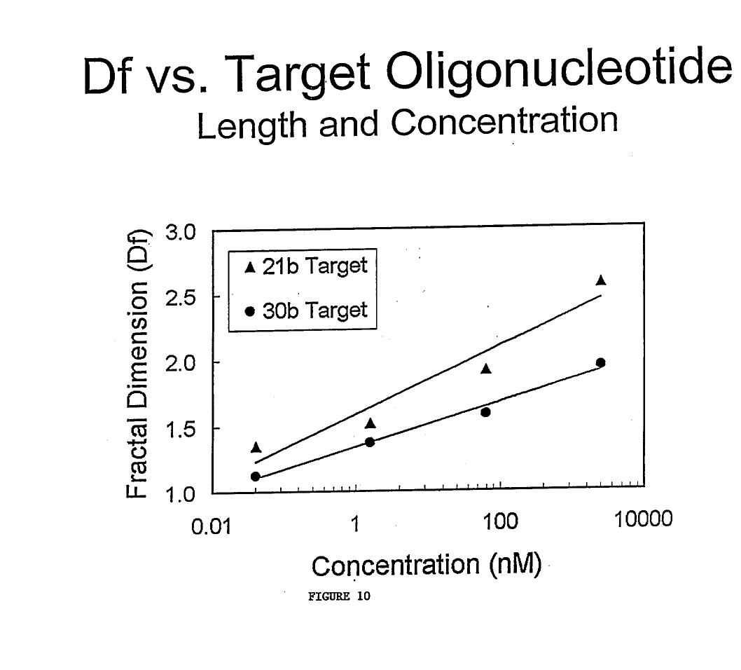

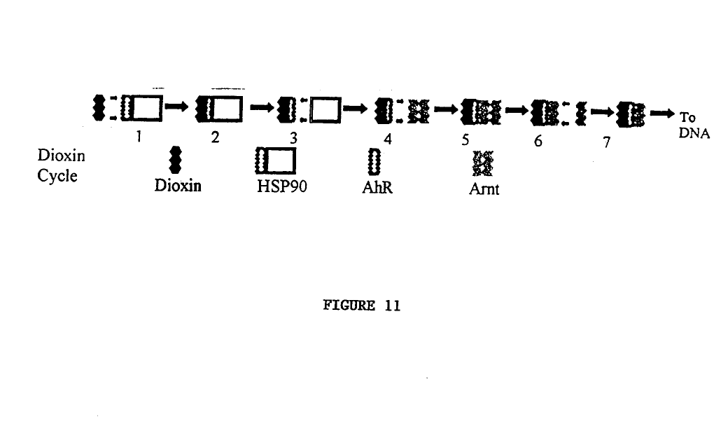

After we determined both, that cystine concentrations higher than 1.56 mM induce aggregation of 40 nm Au, and that concentrations as high as 12.5 mM cystine does not induce aggregation of 100 nm Au nanoparticle, we tested the behavior of unmodified particles in the presence of cystine modified biotin (cyss-biotin). In contrast to the cystine experiment, the highest concentration of cyss-biotin available is 0.450 mM, which is approximately three times lower than the cystine concentration that induced aggregation. This maximum cyss-biotin concentration is defined by the concentration of our stock solution, which is 0.450 mM. During cyss-biotin conjugation, 0.450 mM was the highest concentration that would account for cystine solubility at 12.5 mM and at the same time allowed the use of excess biotin during the synthesis of cyss-biotin; excess biotin is desirable so we can have a high yield of cystine modified with biotin. FIG. 10 and 11 shows the spectra of 40 rnm and 100 nm Au as a function of cyss-biotin concentration. Both sizes of Au nanoparticles showed negligible aggregation, as it was expected based on our results with much higher concentration of unmodified cystine.

Purification of Au-Biotin Probes

After we modified the Au nanoparticles with cyss-biotin, we had to remove the unbound cyss-biotin. The general procedure for the purification procedure of 40 nm Au-biotin comprised of four sequential centrifugation steps (14,000 rpm for 15 minutes), where the supernatant was removed and the sedimented Au is resuspended with equal volume (same volume as initial Au-biotin solution) of nanopure water (repeated 4×). after the final centrifugation step, the final volume of water added is a quarter (25%) of the initial volume, so the final sample is more concentrated than the initial one; a more concentrated purified product is convenient, because it allows one to work with the required small volumes of our scattering cell (200 μL). The purification of the 100 nm nanoparticle does not require high centrifugation speeds; in addition, 100 nm particles can be also purified by sedimentation without centrifugation where 40 nm Au will not sediment (unless undesirable particle aggregation takes place).

The biotin coverage on the Au nanoparticles is next determined. We can qualitatively identify the adsorption of cyss-biotin by comparing the UvVis spectrum of the purified Au-biotin and its first supernatant for 40 nm and 100 nm Au, respectively. Spectrophotometrical methods have limitations, because both biotin and cystine do not absorb in the visible region of light spectrum. The Uv region available to probe these molecules is narrow (200 nm to 280 nm), and this region usually present high absorbance background resulting from many solvents and buffers absorbance at these wavelengths region.

Although there are limitations in using Uv-Vis absorption spectroscopy to characterize the Au-biotin purified product, we still use absorption spectroscopy to qualitatively monitor the reproducibility and consistency of the purified Au-biotin by measuring the absorbance ratio between 270 nm and 514 nm absorption for 40 nm Au, and 260 nm and 564 nm for 100 nm Au; the final purified product should have these ratios of approximately 1 (±0.2) and 1.8 (±0.2) for 40 nm particles and 100 nm, respectively. We determine the presence of biotin on the Au is through function diagnostic using the light scattering approach described herein.

Streptavidin-Streptavidin Conjugation

We used streptavidin and streptavidin complexes to act as bridging molecules between biotin modified Au nanoparticles. We describe the chemistry used to cross-link streptavidin to itself in order to generate streptavidin-streptavidin complexes (streptavidin complex). Historically, the first cross-linking agents used for modification and conjugation of proteins were bireactive molecules carrying the same functional group at both ends. Glutaraldehyde is a popular homobifunctional reagents used for protein conjugation. Amino groups on proteins react with the bis-aldehyde, or glutaraldehyde, to cross-link amino-groups between the proteins.

Generally, when using these homobifunctional cross-linkers for coupling proteins, the main disadvantage is the potential for creating cross-links between two or more proteins, leading to complexes of many sizes. Although this maybe a disadvantage to many applications, this makes glutaraldehyde ideal for this work, since our goal is to create streptavidin-streptavidin complexes of different sizes, such as streptavidin dimers, trimers and larger complexes.

The mechanism of reaction between glutaraldehyde and the primary amines of streptavidin (usually the amine from lysine) is a nucleophilic addition, where the primary amine is the nucleophile which attacks the carbonyl group from glutaraldehyde. The reaction mechanism follow a path which include the formation of a Schiff's base linkage between the aldehyde ends and amines on the protein. Schiff base interactions between aldehydes and amines are usually not stable enough to form irreversible linkages, thus the Schiff's base have to be reduced with suitable reductants, such as sodium borohydride or cyano borohydride. In this work we chose to use of the former; we avoided cyano borohydride because it releases the toxic cyanide gas as one of its by-products (Avrameas 1969; Avrameas 1969; Hermanson, Mallia et al. 1992; Wong 1993; Hermanson 1996). Because of the homobifuntional nature of glutaraldehyde, when one carbonyl end reacts with one streptavidin, it still has the other end which will then cross-link with another streptavidin; this reaction will go on until it is stopped by the addition of an excess of amine carrying molecule, such as glycine, and the reduction of the Schiff's base. It is important clarifying that Streptavidin has approximately 20 lysines available to cross-link with glutaraldehyde (Savage, Mattson et al. 1992).

The procedure used for the. conjugation of streptavidin to itself was adapted from Bioconjugate Techniques by Hermanson (Hermanson 1996), as it is described: first, streptavidin (from Sigma) was solubilized in 0.1 M sodium bicarbonate to 20 mg/ml. After solubilization, streptavidin was further quantitated using spectrophotometric absorption at 280 nm; the extinction coefficient for streptavidin at 280 nm is 3.2 au/mg (Savage, Mattson et al. 1992). The streptavidin concentration was adjusted to 10 mg/ml with 0.1 M sodium bicarbonate. For 1 ml of streptavidin solution at 10 mg/ml, 30 μl of 2.5% of glutaraldehyde was added. After 30 minutes from the addition of glutaraldehyde, 50 μl of 1.0 M glycine is added to block unreacted carbonyl groups; and 0.166 ml of 10 mg/ml of fresh sodium borohydride (NaBH₄) were added and allowed to react for 10 minutes for the reduction of the Schiff's base. Finally, 38 μl of 4M acetic acid was added to reduce the pH of the final solution. After complexation procedure was complete, the protein solution was dialyzed in borate buffer to remove unreacted small molecular weight reagents and by-products, and for buffer exchange from sodium carbonate to borate buffer. The three HPLC chromatograms in FIG. 15 show the chromatograms of the streptavidin-complex, streptavidin monomer and their controls, where the controls are the same samples saturated with unmodified biotin.

The concentration of the complex and monomer that were used in the experiments described in the following sections were adjusted to the same optical density value measured at 280 nm wavelength of light. The fact that both the monomer and the complex have the same absorbance values, translates to the fact that there is approximately the overall mass of protein in both samples, but the actual number of individual units is lower for the complex.

Scattering Measurements

We investigated the scattering signal, Df and Rg sensitivity to the concentration of a streptavidin-complex interacting with Au modified with biotin. The angle dependent scattering signal is sensitive to the concentration of streptavidin-complex. This sensitivity is specially pronounced at forward scattering angles. This is a result of the aggregation process where the scattering signal is dependent in the number of particles forming in an aggregate, as shown below (Farias, Koylu et al. 1996),

C_{sca,a}(θ)∝N²·C_{sca,p}

where the cross-section of scattering as a function of scattering angle (C_{sca,a}(θ)) is proportional to the square of number of particles (N) times the cross-section of scattering for the individual particles. The scattering signal for the control streptavidin-complex sample is not sensitive to the control concentration (streptavidin complex saturated with unmodified biotin). The fractal dimension analysis of the scattering results where the negative of the slope yields the fractal dimension. The results show that both the radius of gyration and the fractal dimension are found to be sensitive to target concentration. However, the latter shows a greater sensitivity. The increase in aggregate density (as shown by the increase in Df with streptavidin-complex target concentration) is likely the outcome of increased intra-particle cross-linking at higher streptavidin-complex concentration. These results also show that the control (streptavidin-complex incubated with free biotin) remains nearly unchanged for the various streptavidin complex control concentrations.

We also compared the fractal dimension between aggregates assembled with streptavidin-complex and aggregates assembled with streptavidin monomer. During this study we used 532 nm and 632 nm incident light, where the results for measurements using both wavelengths were in agreement. The data suggest that the morphology of the aggregates is a function of concentration and structure composition of the target protein. We measured the fractal dimension values for two systems with different target proteins: one where the protein target is a streptavidin monomer, and the other where the target protein is the streptavidin complex (already described). Both protein targets present the same function, but the protein complex is a mixture of streptavidin complexes. Each concentration of target protein showed higher Df values for the system where the streptavidin monomer was the target, in comparison to the streptavidin complex. This shows that the larger protein targets, which make up the streptavidin complex, assemble the Au nanoparticles into more swollen and better solvated aggregates. Further, the increase in fractal dimension with concentration for both types of protein targets further suggests a transition from string like aggregates (when Df is near unity) to denser, more compact structures at higher fractal dimensions.

We also observed that the difference in fractal dimension between the two systems is more pronounced at lower concentrations than higher concentrations. The results suggest that the difference in fractal dimension sensitive to both concentration and protein target structure.

Antibody Detection

We applied angle dependent light scattering and fractal dimension to detect the reactivity of 40nm Au nanoparticles modified with goat antibodies towards a target antigen. The Au nanoparticles were modified with either goat anti-mouse immunoglobulin (Gt anti-Ms IgG) or goat anti-human immunoglobulin (Gt anti-Hu IgG); the target antigen was mouse immunoglobulin G protein (Mouse IgG), which should be reactive mainly with Gt anti-Ms IgG. Since an IgG has two binding sites available to interact with an antigen, we anticipated that the presence of a target antigen would trigger the aggregation of the antibody modified Au nanoparticles, forming an aggregate of nanoparticle, antibody and antigen. Our results were in agreement with our predictions. The nanoparticles carrying the Gt anti-Ms IgG antibody showed the highest reactivity towards the target mouse IgG antigen. The sample with no antigen showed low forward scattering signal, because no specific aggregation should have taken place. However, the system where Au nanoparticles were modified with Gt anti-Hu showed an increased forward scattering signal, which indicates cross reactivity between the Gt anti-Hu IgG antibody towards mouse IgG.

The fractal dimension analysis and Guinier analysis also showed that the sample with the highest reactivity towards the target antigen had both, highest fractal dimension and largest radius of gyration. As expected, the higher reactivity resulted in an increase in interparticle cross-linking, which originated more compact aggregates with larger fractal dimension. The larger radius of gyration is also the outcome of having higher reactivity, which then result in a larger number of particles in an aggregate.

This method can be further adapted towards the determination of antibody titer towards a specific antigen. Because antibodies are being routinely developed to be used in drug discovery as drug delivery and diagnostic agents, the determination of not only the reactivity with its target antigen is necessary, but also the measurement of its cross reactivity with other proteins is required. The Au nanoparticles modified with Gt anti-Hu showed cross reactivity towards the mouse IgG. Because of the common mammalian denomination of humans and mouse, Gt anti-Hu antibodies still show cross reactivity to mouse IgG, which our results also confirm.

Our sample comprised of 1:5 dilution of 40 nm modified Au nanoparticle stock solution from KPL in the presence of 10 μg/ml of Mouse IgG (antigen) to a total solution volume of 500 μL. Control 1 (blue) contained mouse IgG as the target antigen and 40 nm Au modified with Gt anti-Hu; control 2 (red) did not have any antigen, it consisted of 40 nm Au modified with Gt; the test (green) had mouse IgG as target antigen and 40 nm Au modified with Gt anti-Ms IgG.

Au-Biotin and Au-Streptavidin

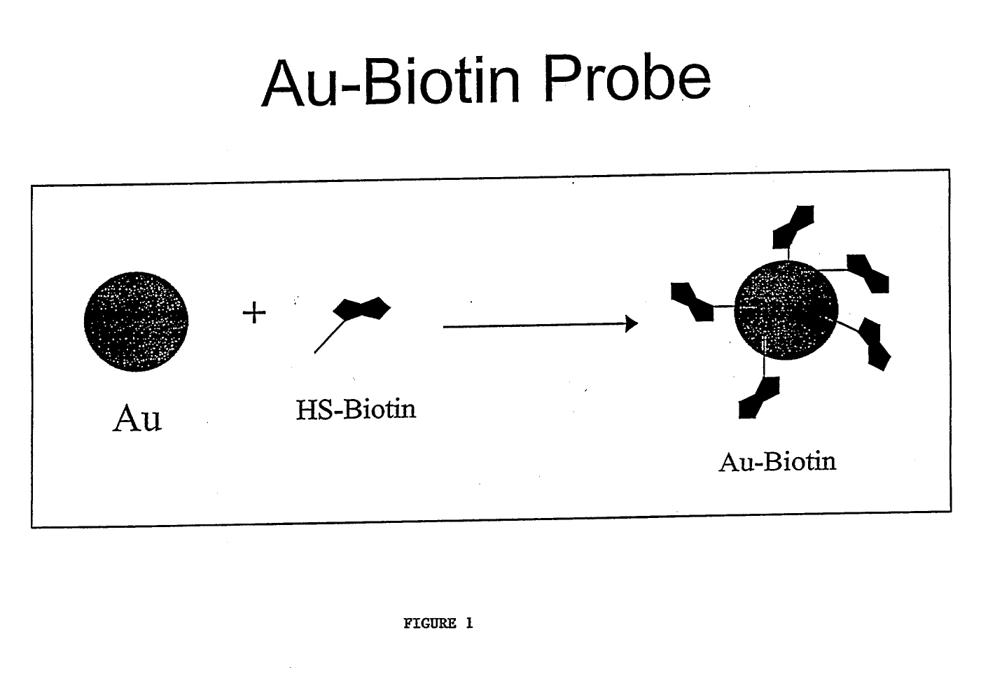

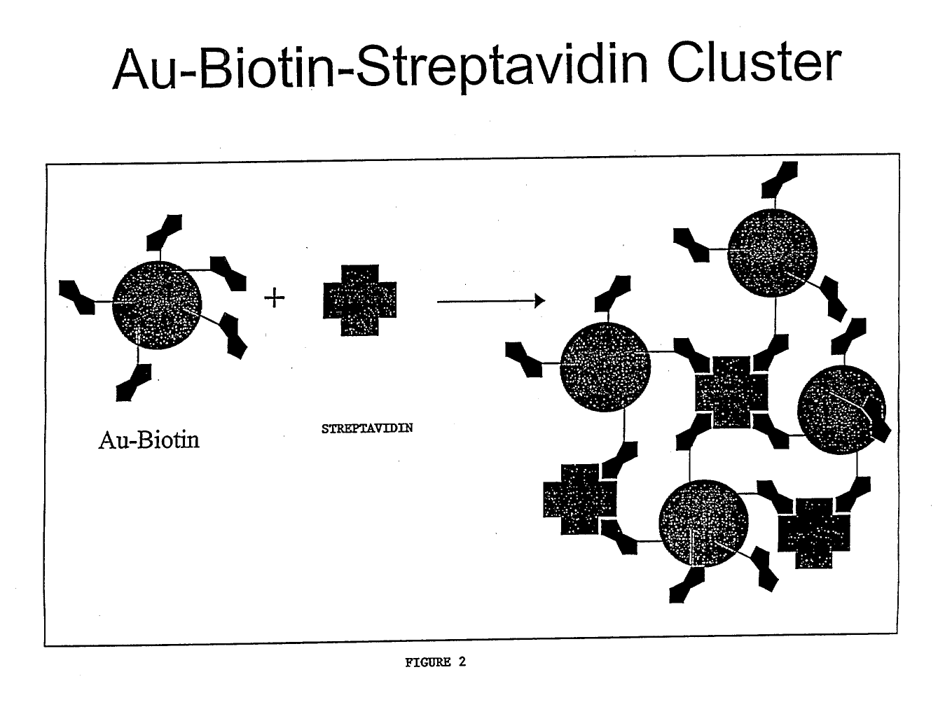

The methods of the present invention employing ADLS/FD are used to probe the binding of biotin modified gold nanoparticles to streptavidin in a liquid sample. Fractal dimension analysis of ADLS signal provide information about the size and structure of Au-Protein aggregates which allow us to further infer the structural characteristics of the binding protein. FIG. 1 shows the Au-Biotin Protein and FIG. 2 shows the Au-Biotin-Streptavidin cluster of this invention.

Streptavidin (Savage, 1992) is a protein which has four binding sites available to bind biotin. The streptavidin-biotin bond has a dissociation constant of 10⁻¹⁵ M. The streptavidin-biotin complex is an ideal model system because the strong biotin streptavidin bond and the four available binding sites allow streptavidin to be used as a linker between biotin modified gold nanoparticles (Au-Biotin probe) forming a fractal network of Au and protein (Au-Protein aggregate). Au-Biotin probes were made by labeling thiol-modified biotin with gold nanoparticles of specific sizes as known by those skilled in the art (Mirkin, 1996; Elghanian, 1997).

We detected the binding of biotin to streptavidin by monitoring changes in the angle-dependent scattering signal produced by the Au-Biotin probe as it binds to streptavidin. The fractal dimension of the Au-Protein aggregates was extracted from the analysis of the ADLS signal (Avnir, 1984). Because the fractal dimension of the Au-Protein aggregates depends on the size, shape and number of binding sites of the linking proteins, fractal dimension of these Au-Protein aggregates were correlated to the structural characteristics of the proteins. The method of the present invention includes applying the method to systems where proteins bind to specific molecules, such as small molecular weight molecules and other proteins (or peptides), by modifying Au nanoparticles with such molecules. This method is an excellent tool for detection of cancer related proteins, for rapid characterization of proteins and for drug targeting studies.

Dioxin Detection

Dioxins represent a family of environmental pollutants which can cause severe health risk, from cancer to developmental defects in several species. The nature of dioxins and related compounds is to bind to aromatic hydrocarbon receptors (AhR), part of a heat shock protein (HSP90 protein, MW=50 kDa) as shown in FIG. 11. After binding to AhR, this receptor is translocated and it binds to AhR nuclear translocator receptor (Arnt), a dimmer. Upon binding, the Arnt-dioxin complex splits and the dioxin monomer complex then binds to DNA.

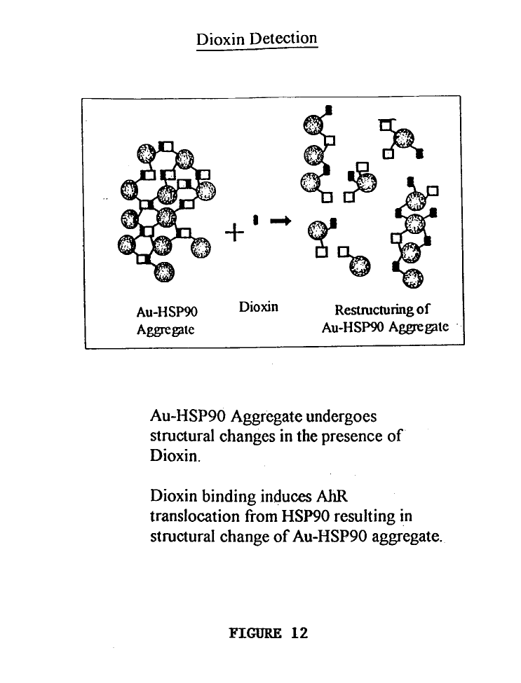

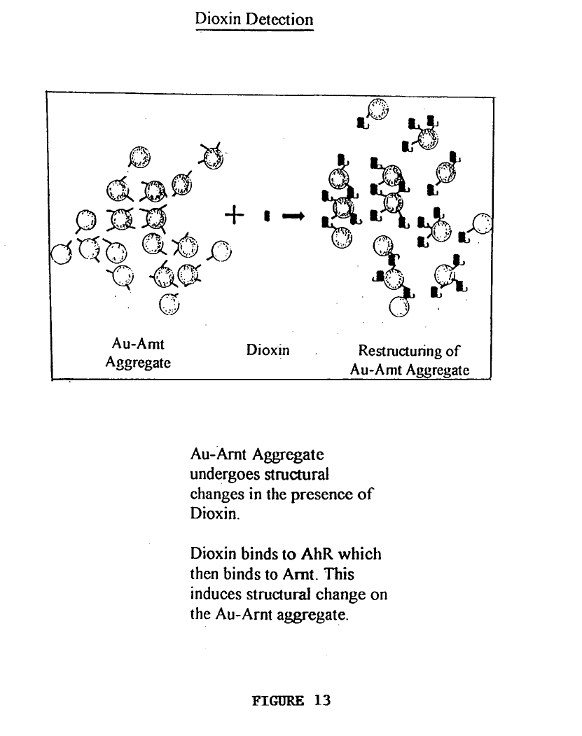

The methods of the present invention include using Au-protein (HSP90, Arnt or AhR) aggregates for detecting dioxin. The method includes constructing Au-protein aggregates using HSP90, Arnt or AhR as linkers between the Au nanoparticles. FIGS. 12 and 13 show two examples of Au-Protein aggregates for detecting dioxin according to the present invention. The Au-Protein aggregate undergoes significant structural changes upon binding to dioxin. The Au-Protein aggregates will be formed by using heterobifunctional crosslinkers to anchor the proteins to the Au nanoparticles. A dioxin biosensor is provided that detects analogous aggregate changes as dioxin binds to HSP90.

In one example of the invention, the ADLS apparatus was constructed from commercially available optical components. Hybridization samples comprised 0.1 pM of Au-DNA probe and oligo-target concentrations ranged from about 0 nM to 2500 nM.

The Au-DNA probes were prepared by mixing 9.0 pM of citrate stabilized 100 nm gold nanoparticles (purchased from BB International) with 5 nM of either 5′ or 3′ end hexanethiol modified oligonucleotide (HS(CH2)6-CCC-GCG-CCC-3′ [SEQ ID NO:3] and 5′-CCC-GCG-CCC-(CH2)6SH [SEQ ID NO:3], respectively, without addition of salt and by incubation for 24 hours at 4° C. Au-DNA probes were purified by centrifugation (14,000g for 15 minutes), where the supernatant was discarded and the precipitate, Au-DNA probes, were resuspended in hybridization buffer. All oligonucleotides were purchased from Genset Corp.

The hybridization buffer was comprised of 50% (by volume) 5OmM borate buffer at pH 8.6 and 50% 10 mM TE (10 mM Tris+1 mM EDTA) TE buffer pH 8.0. Borate was used because modified and bare gold nanoparticles have shown greater stability in sodium borate buffer than in the presence of other salts, such as sodium phosphate and sodium chloride. TE buffer was chosen because it is routinely used by molecular biologists when manipulating DNA. The concentration of Au-DNA probes was determined by absorbance measurements at 575 nm using an extinction coefficient of 1.62×10¹¹ M⁻¹ cm⁻¹.

The oligonucleotide targets, 5′-GGG-CGC-GGG-ATA-GGG-CGC-GGG-3′ [SEQ ID NO: 1] and 5′-GGG-CGC-GGG-AAA-TAA-AAT-AAA-GGG-CGC-GGG-3′ [SEQ ID NO: 2], comprise synthesized oligonucleotides of either 21 or 30 bases in length, where the nine-base sequence on the 3′- and 5′- ends were complementary to the sequences of the Au-DNA probes. The present invention provides that an aggregate of Au-DNA nanoparticles assembled by the hybridization of AU-DNA probes to their oligo-target is an example of fractal structure.

Hybridization procedure for each target consisted of three heat and cool cycles in which the targets were heated to 70° C. in a water bath for 10 minutes and then cooled in an ice bath for anther ten minutes. After the third cycle of heating and cooling, each target was incubated for 24 hours at 4° C. Each target was removed from the 4° C. environment just prior to ADLS measurement; for each ADLS measurement, 100 μL of the sample was extracted and mixed with 900 μL of hybridization buffer.

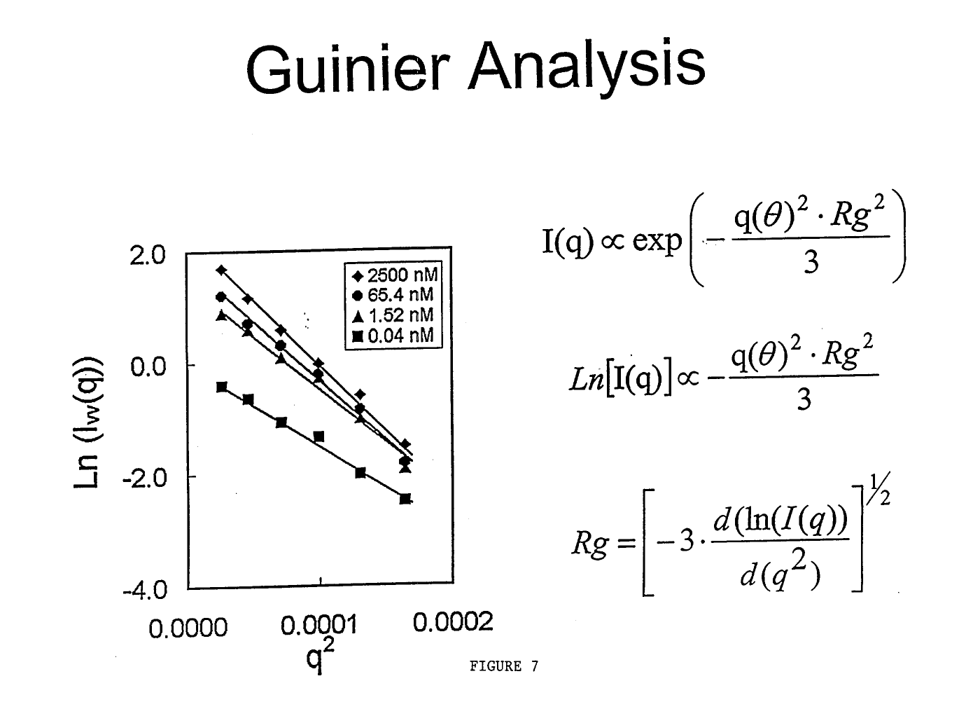

FIG. 6 shows the scattering signals for 30°≦θ≦150° for the Au-biopolymer hybridization with different concentrations of oligo-target molecule. Because the scattering intensity integrated across all angles increases with target concentrations, these results are consistent with published literature results, where UV-vis measurements have shown that extinction increases as a function of target concentration. See Mirkin, supra. As suggested above, analysis of the angle dependence of the scattering signal can be used to calculate both Rg and Df (see Equations 1 and 2), and thus unveil details of the aggregate morphology. A Guinier plot allows the determination of the radius of gyration of an aggregate from the analysis of scattering intensity, I(q), at low scattering angles. See FIG. 7. Under these conditions:

$$I ( q ) ∝ exp ( - \frac{{q ( θ )}^{2} \cdot {Rg}^{2}}{3} ) \qquad \text{(Equation 3)}$$

Therefore, Rg may be obtained from the slope of a plot of the natural logarithm of I(q) as a function of the square of the scattering wavevector:

$$Rg = {[ - 3 \cdot \frac{ⅆ ( ln ( I ( q ) )}{ⅆ ( {q}^{2} )} ]}^{\frac{1}{2}} \qquad \text{(Equation 4)}$$

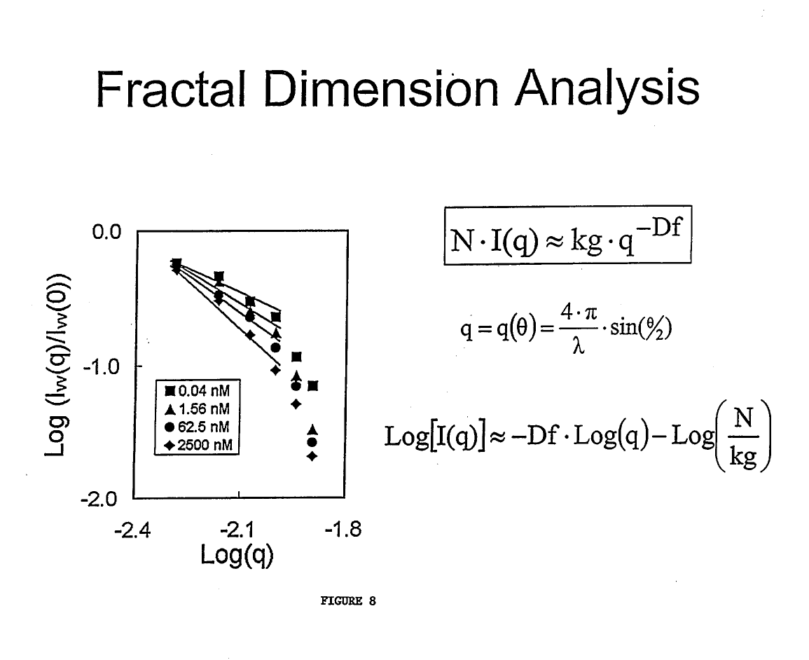

The fractal dimension (Df) can also be extracted from the angle-dependent scattering data. From Equation 2 it can be seen that the negative of the slope of the logarithm of the normalized angle-dependent scattering signal versus the logarithm of the wavevector q(θ) yields the fractal dimension. This relationship is only valid at small angles, where q(θ) is less than or equal to 0.010 nm⁻¹. See FIG. 8.

The ADLS measurements were performed, and data was collected for angles where 30°≦θ≦150°. This data was used to determine both Rg and Df of the samples, and therefore allowed the unveiling of certain details of the aggregate morphology. Table 1 summarizes the results of the analyses performed on the data presented in FIGS. 7, 8 and 9. Both the radius of gyration and the fractal dimension were found to be sensitive to target concentration, with the later having a greater sensitivity. While not being bound to the following theory, the applicants believe that the increase in aggregate density (as shown by the increase in Df with oligo-target concentration) is due to the outcome of increased intraparticle cross-linking at higher oligo-target concentration. The exponential difference of the signal intensity on Df (Equation 2) makes the method of the present invention very sensitive and therefore ideal for detecting an oligo-target DNA fragment using Au-DNA probes.

The data presented in FIGS. 7, 8 and 9 suggest that the morphology of the aggregates can be measurably altered by target concentration. Table 1 shows that both Df and Rg increase with target concentration.

TABLE 1

| Target | | |
|---|---|---|
| Concentration | | |
| (nM) | Rg (nm) | Df |
| 0.04 | 215 | 1.3 |
| 1.56 | 245 | 1.6 |
| 62.5 | 251 | 1.9 |
| 2500 | 260 | 2.5 |

FIG. 10 shows that the fractal dimension is also dependent on the length of the oligo-target, effectively increasing the spacing between primary particles sensitive to the length of the target oligonucleotide. FIG. 10 shows a comparison of the fractal dimension values for two systems with different oligo-target lengths: one in which the Au-DNA probe hybridizes to a 21-base long oligo-target, and the other in which the Au-DNA probe hybridizes to a 30-base long oligo-target. Each concentration of target tested showed lower Df values for the system containing the 30-base long oligonucleotide target, in comparison to the one containing the 21 -base long oligonucleotide target, indicating, more swollen and better solvated clusters for the former. Further, the increase in fractal dimension with concentration for both lengths of oligo-target suggests that a transition from string like aggregates (when Df is near unity) to denser, more compact structures at higher fractal dimension.

It will be appreciated by those persons skilled in the art that the present invention provides a method to detect small structural variations as Au-DNA nanoparticles assemble into aggregates. The interference between scattered electromagnetic waves from the particles within the fractal aggregate is a factor in calculating the fractal dimension (Df) of the aggregate, a value that is simultaneously sensitive to both the concentration and length of an oligonucleotide target. The present invention provides an apparatus for light scattering that delivers both faster and superior angle resolution than traditional light scattering methodologies, and that is incorporated into a portable instrument. The present invention provides a method for performing in situ analysis of nanostructure formation dynamics and morphology in a liquid environment. It will be understood by those persons skilled in the art that the present invention employs biological molecules as building blocks for building nanoscale structures.

Whereas, particular embodiments of this invention have been described for purposes of illustration, it will be evident to those persons skilled in the art that numerous variations of the details of the present invention may be made without departing from the invention as defined in the appended claims and SEQUENCE LISTING.

We claim:

1. A method for determination of at least one property of a test sample, the method comprising the steps of:
providing a test sample;
suspending said test sample in a first hybridization buffer;
preparing at least one nanoparticle-biopolymer probe;
suspending at least one of said nanoparticle-biopolymer probe in a second hybridization buffer;
combining said test sample and said first hybridization buffer solution with said nanoparticle-biopolymer probe and said second hybridization buffer solution;
effecting hybridization of at least one of said nanoparticle-biopolymer probe with said test sample, creating a test aggregate;
suspending said test aggregate in a third hybridization buffer;
placing said test aggregate in said third hybridization buffer solution in an angle dependent light scattering apparatus;
operating said angle dependent light scattering apparatus;
detecting and recording the results of said operation of said angle dependent light scattering apparatus;
using said results to calculate at least the fractal dimension of said test aggregate; and
comparing said fractal dimension with known values to determine at least one property of said test sample.

2. The method of claim 1, wherein said nanoparticle-biopolymer probe is comprised of at least one gold (Au) nanoparticle bonded to at least one thiol-modified oligonucleotide.

3. The method of claim 1, wherein the test sample is comprised of at least one of a DNA, a RNA, or a protein.

4. The method of claim 1, including wherein said property of a test sample comprises one or more of a base length of the oligonucleotide of said test sample and concentration of said oligonucleotide of said test sample.

5. An angle dependent light scattering apparatus comprising:
at least one electromagnetic beam;
at least one polarizer for polarizing said electromagnetic beam;
a lens and an iris for focusing said polarized electromagnetic beam;
a scattering cell for receiving said polarized electromagnetic beam, wherein said scattering cell is capable of containing a sample solution through which said polarized electromagnetic beam is passed for producing scattered light;
at least one collection device for collecting and collimating said scattered light;
a second polarizer for isolating said collimated and scattered light;
an another lens for focusing said collimated and scattered light onto a plane into at least one analysis device for analyzing said data; and
at least one computer for coordinating the control of the operation of said angle dependent light scattering apparatus, wherein at least said scattering cell and said collection device are on a stage capable of rotation.

6. The apparatus of claim 5, wherein said electromagnetic beam is emitted from a photodiode laser head.

7. The apparatus of claim 6, wherein said photodiode laser head has an output wavelength of about 660 nm.

8. The apparatus of claim 6, wherein said photodiode laser head is mounted in a cooled mount.

9. The apparatus of claim 5 wherein said scattering cell comprises an outer reservoir including a sealed first end and a second end having a removable cap that is in sealing engagement with said second end of said outer reservoir, and an inner sample reservoir located within said tubular outer reservoir, wherein said inner sample reservoir has a sealed first end and a second end and a body disposed between said first end and said second end of said inner sample reservoir, and wherein said second end of said inner sample reservoir is in juxtaposition to said removable cap of said outer reservoir.

10. The apparatus of claim 5, wherein said stage is controlled by said computer for operating the angle dependent light scattering apparatus.

11. The apparatus of claim 5 including a thermoelectric device in contact with said scattering cell for regulating and detecting the temperature of said sample solution and regulating the temperature of said sample solution.

12. The apparatus of claim 5 including a interruption device for accomplishing repeated interruption of said electromagnetic beam at designated intervals.

13. The apparatus of claim 5 including a beam blocker placed in juxtaposition to said scattering cell to prevent excess light from escaping said scattering cell.

14. The apparatus of claim 5 wherein said analysis device is a charge-coupled device array detector.

15. An angle dependent light scattering apparatus comprising:
at least one electromagnetic beam;
at least one polarizer for polarizing said electromagnetic beam;
a lens and an iris for focusing said polarizing electromagnetic beam;
a scattering cell for receiving said polarized electromagnetic beam, wherein said scattering cell is capable of containing a sample solution through which said polarized electromagnetic beam is passed to produce scattered light;
at least one collection device for collecting said scattered light;
a second polarizer for isolating said scattered light;
a charge-coupled device array detector for collecting the image of said polarized scattered light; and
at least one computer for coordinating the control of the operation of said angle dependent light scattering apparatus.

16. The angle dependent light scattering apparatus of claim 15 wherein said collection device is an ellipsoidal mirror.

17. An aggregate cluster comprising a gold-biopolymer probe hybridized to a target molecule wherein said gold-biopolymer probe comprises a gold nanoparticle attached to a modified biopolymer.

18. The aggregate cluster of claim 17 wherein said gold nanoparticle has a diameter ranging from about 1 nanometer to greater than 100 nanometers.

19. The aggregate cluster of claim 18 wherein said gold nanoparticle has a diameter ranging from about 50 nanometers to about 300 nanometers.

20. The aggregate cluster of claim 17 wherein said biopolymer is a oligonucleotide, a DNA, a RNA or a protein.

21. The aggregate cluster of claim 20 wherein said oligonucleotide has a base pair length of about less than or equal to eighty base pairs.

22. The aggregate cluster of claim 17 wherein said biopolymer is a biotin.

23. The aggregate cluster of claim 17 wherein said target molecule is a nucleic acid.

24. The aggregate cluster of claim 17 wherein said target molecule is an oligonucleotide.

25. The aggregate cluster of claim 17 wherein said target molecule is DNA, RNA or a protein.

26. The aggregate cluster of claim 17 wherein said target molecule is streptavidin.

27. The aggregate cluster of claim 17 wherein said target molecule is dioxin.

28. The aggregate cluster of claim 17 wherein said target molecule is a modified oligonucleotide.

29. The aggregate cluster of claim 17 wherein said modified oligonucleotide has the SEQ ID NO: 3.

30. The method of claim 1 including employing said method for monitoring formation of nanostructure.

31. A biosensor comprising a gold-biopolymer nanoparticle probe and an angle dependent light scattering detection device wherein said gold-biopolymer nanoparticle probe is capable of interacting with other biopolymers or molecules.

32. The biosensor of claim 31 wherein said angle dependent light scattering detection device is said apparatus of claim 15.

33. A scattering cell comprising an outer reservoir including a sealed first end and a second end, and a body disposed between said first and said second ends of said outer reservoir, wherein said second end of said outer reservoir having a removable cap that is in sealing engagement with said second end of said outer reservoir, and an inner sample reservoir located within said outer reservoir, wherein said inner sample reservoir has a sealed first end, a second end, and a body disposed between said first end and said second end of said inner sample reservoir, wherein said second end of said inner sample reservoir is in juxtaposition to said removable cap of said outer reservoir.

34. The scattering cell of claim 33 including wherein said removable cap has a sample inlet that is in communication with said inner sample reservoir.

35. The scattering cell of claim 33 including wherein said removable cap has a second inlet that is in communication with said outer reservoir.

36. The scattering cell of claim 33 wherein said outer reservoir and said inner reservoir each have a tubular shape.
